SEQUENCE LISTING (36 sequences)

SEQ ID NO: 1 - DNA, 13 bp, Tomato golden mosaic virus
ggtagtaaggtag

SEQ ID NO: 2 - DNA, 16 bp, bean yellow dwarf virus
tggaggcatggaggca

SEQ ID NO: 3 - DNA, 12 bp, bean yellow dwarf virus
ttccttacctat

SEQ ID NO: 4 - DNA, 18 bp, bean yellow dwarf virus
cactatcatgctcttctc

SEQ ID NO: 5 - DNA, 21 bp, bean yellow dwarf virus
gtccttgattacatatcaaag

SEQ ID NO: 6 - DNA, 21 bp, Tomato golden mosaic virus
tttcttacatatcctcagtgc

SEQ ID NO: 7 - DNA, 21 bp, Tomato golden mosaic virus
cacctccacgtgcttattcag

SEQ ID NO: 8 - DNA, 21 bp, Tomato golden mosaic virus
gtcaagacgtacatcgacaaa

SEQ ID NO: 9 - DNA, 23 bp, bean yellow dwarf virus
gcgacaagggggggcccacgccg

SEQ ID NO: 10 - DNA, 26 bp, cabbage leaf curl virus
atgtatcctacaaagtttaggcgtgg

SEQ ID NO: 11 - DNA, 24 bp, cabbage leaf curl virus
ctatgtaattaaacgcatttggag

SEQ ID NO: 12 - DNA, 13 bp, Tomato golden mosaic virus
ggtagtaaggtag

SEQ ID NO: 13 - DNA, 16 bp, bean yellow dwarf virus
tggaggcatggaggca

SEQ ID NO: 14 - DNA, 12 bp, bean yellow dwarf virus
ataggtaaggaa

SEQ ID NO: 15 - DNA, 21 bp, bean yellow dwarf virus
ctggagaagagcatgatagtg

SEQ ID NO: 16 - DNA, 21 bp, bean yellow dwarf virus
ctttgatatgtaatcaaggac

SEQ ID NO: 17 - DNA, 15 bp, bean yellow dwarf virus
ttcgtggcagcgaag

SEQ ID NO: 18 - DNA, 23 bp, bean yellow dwarf virus
gcgacaagggggggcccacgccg

SEQ ID NO: 19 - DNA, 26 bp, cabbage leaf curl virus
atgtatcctacaaagtttaggcgtgg

SEQ ID NO: 20 - DNA, 20 bp, cabbage leaf curl virus
ctatgtaattaaacgcattt

SEQ ID NO: 21 - DNA, 13 bp, Tomato golden mosaic virus
ctaccttactacc

SEQ ID NO: 22 - DNA, 16 bp, bean yellow dwarf virus
tgcctccatgcctcca

SEQ ID NO: 23 - DNA, 12 bp, bean yellow dwarf virus
ttccttacctat

SEQ ID NO: 24 - DNA, 21 bp, bean yellow dwarf virus
cactatcatgctcttctccag

SEQ ID NO: 25 - DNA, 21 bp, bean yellow dwarf virus
gtccttgattacatatcaaag

SEQ ID NO: 26 - DNA, 15 bp, bean yellow dwarf virus
cttcgctgccacgaa

SEQ ID NO: 27 - DNA, 23 bp, bean yellow dwarf virus
cggcgtgggcccccccttgtcgc

SEQ ID NO: 28 - DNA, 26 bp, cabbage leaf curl virus
ccacgcctaaactttgtaggatacat

SEQ ID NO: 29 - DNA, 20 bp, cabbage leaf curl virus
aaatgcgtttaattacatag

SEQ ID NO: 30 - DNA, 1366 bp, cotton leaf curl virus
accgtgggcgagcggtgtctttggcgttccatgtgggtcccacaatatccaaaagaagaataatggactgggtcaatgcaattgggccttaaatgaaatgggcttggaccagtagattcgagactgggccaatagaataaaacaacaaatggactcataatcaaaacaaagtgtttattcatgtcaaatacattacacactcacacacacacagtcgtacacacatcatattcatcccctatacgtatatcaactaatggggcctcatgcatcatcattatatcaatagcctctaccatgtcctcctggcgaaagtcccgaacatgacaatccctatacatgatctttaatatattatgtatcccttcctccaaattgttgaagtcgaaaggcggtatgatcccatcatggccgtatgggatcatgaaggtcttctttgccagggcaggtgatcttgttgagcacaattcaatccgcacaaggattgaattgtcctcgtggatcttcacgtcgacggtaaataccatccccttctcgttagtatacttgattgtcatttgcatttaattatgaacaacacatgagatgaatcttcttaaatagcgtccatatatttggatatatggacataatgcatatacgtggtgcaataattatcatatgaatatgagtggagacatatatgattgatatctacaggactatcaatcatcgtcaagaaaaaagggataaaagaaaccttattcatgaaaggattaggtagggaaaaataaaagaaggaaattaaggaaaagaaaacccacaatgaaataattaagaaaaaaaaaagaaaaaaagaacacaagaaaggaaaacagaaacatgagcacaaacagaaacctcttgagaaaatattggagcgcagcgggaaaaccaagaaaaacaaaccaaggaagacacttgagcaaaaagggaaaacacaaactaaactaggagggtccaacatgaaattatgaaaagtccgtacacagtaattaatcattaattactgcgcagtaaatgtgaataaaattaacccgaagggttaattttcgagaccccgataggtaattgagtccccaatatatcggggactcaatcggggtcatgagagagaaataaatcccggataccgaaactaccctcaaagctgtgtctggaaggcgcgtgggagtgcgctgaaaaaggtgaccttctctctccaaaaactcaccggaacggccaaactggctgattccggcatctaatcacgacacgcgcggcggtgtgtacccctgggagggtagaccactacgctacgcagcagccttagctacgccggagcttagctcgcccacgttctaatatt

SEQ ID NO: 31 - DNA, 4156 bp, Streptococcus pyogenes
atggataagaaatactcaataggcttagatatcggcacaaatagcgtcggatgggcggtgatcactgatgaatataaggttccgtctaaaaagttcaaggttctgggaaatacagaccgccacagtatcaaaaaaaatcttataggggctcttttatttgacagtggagagacagcggaagcgactcgtctcaaacggacagctcgtagaaggtatacacgtcggaagaatcgtatttgttatctacaggagattttttcaaatgagatggcgaaagtagatgatagtttctttcatcgacttgaagagtcttttttggtggaagaagacaagaagcatgaacgtcatcctatttttggaaatatagtagatgaagttgcttatcatgagaaatatccaactatctatcatctgcgaaaaaaattggtagattctactgataaagcggatttgcgcttaatctatttggccttagcgcatatgattaagtttcgtggtcattttttgattgagggagatttaaatcctgataatagtgatgtggacaaactatttatccagttggtacaaacctacaatcaattatttgaagaaaaccctattaacgcaagtggagtagatgctaaagcgattctttctgcacgattgagtaaatcaagacgattagaaaatctcattgctcagctccccggtgagaagaaaaatggcttatttgggaatctcattgctttgtcattgggtttgacccctaattttaaatcaaattttgatttggcagaagatgctaaattacagctttcaaaagatacttacgatgatgatttagataatttattggcgcaaattggagatcaatatgctgatttgtttttggcagctaagaatttatcagatgctattttactttcagatatcctaagagtaaatactgaaataactaaggctcccctatcagcttcaatgattaaacgctacgatgaacatcatcaagacttgactcttttaaaagctttagttcgacaacaacttccagaaaagtataaagaaatcttttttgatcaatcaaaaaacggatatgcaggttatattgatgggggagctagccaagaagaattttataaatttatcaaaccaattttagaaaaaatggatggtactgaggaattattggtgaaactaaatcgtgaagatttgctgcgcaagcaacggacctttgacaacggctctattccccatcaaattcacttgggtgagctgcatgctattttgagaagacaagaagacttttatccatttttaaaagacaatcgtgagaagattgaaaaaatcttgacttttcgaattccttattatgttggtccattggcgcgtggcaatagtcgttttgcatggatgactcggaagtctgaagaaacaattaccccatggaattttgaagaagttgtcgataaaggtgcttcagctcaatcatttattgaacgcatgacaaactttgataaaaatcttccaaatgaaaaagtactaccaaaacatagtttgctttatgagtattttacggtttataacgaattgacaaaggtcaaatatgttactgaaggaatgcgaaaaccagcatttctttcaggtgaacagaagaaagccattgttgatttactcttcaaaacaaatcgaaaagtaaccgttaagcaattaaaagaagattatttcaaaaaaatagaatgttttgatagtgttgaaatttcaggagttgaagatagatttaatgcttcattaggtacctaccatgatttgctaaaaattattaaagataaagattttttggataatgaagaaaatgaagatatcttagaggatattgttttaacattgaccttatttgaagatagggagatgattgaggaaagacttaaaacatatgctcacctctttgatgataaggtgatgaaacagcttaaacgtcgccgttatactggttggggacgtttgtctcgaaaattgattaatggtattagggataagcaatctggcaaaacaatattagattttttgaaatcagatggttttgccaatcgcaattttatgcagctgatccatgatgatagtttgacatttaaagaagacattcaaaaagcacaagtgtctggacaaggcgatagtttacatgaacatattgcaaatttagctggtagccctgctattaaaaaaggtattttacagactgtaaaagttgttgatgaattggtcaaagtaatggggcggcataagccagaaaatatcgttattgaaatggcacgtgaaaatcagacaactcaaaagggccagaaaaattcgcgagagcgtatgaaacgaatcgaagaaggtatcaaagaattaggaagtcagattcttaaagagcatcctgttgaaaatactcaattgcaaaatgaaaagctctatctctattatctccaaaatggaagagacatgtatgtggaccaagaattagatattaatcgtttaagtgattatgatgtcgatcacattgttccacaaagtttccttaaagacgattcaatagacaataaggtcttaacgcgttctgataaaaatcgtggtaaatcggataacgttccaagtgaagaagtagtcaaaaagatgaaaaactattggagacaacttctaaacgccaagttaatcactcaacgtaagtttgataatttaacgaaagctgaacgtggaggtttgagtgaacttgataaagctggttttatcaaacgccaattggttgaaactcgccaaatcactaagcatgtggcacaaattttggatagtcgcatgaatactaaatacgatgaaaatgataaacttattcgagaggttaaagtgattaccttaaaatctaaattagtttctgacttccgaaaagatttccaattctataaagtacgtgagattaacaattaccatcatgcccatgatgcgtatctaaatgccgtcgttggaactgctttgattaagaaatatccaaaacttgaatcggagtttgtctatggtgattataaagtttatgatgttcgtaaaatgattgctaagtctgagcaagaaataggcaaagcaaccgcaaaatatttcttttactctaatatcatgaacttcttcaaaacagaaattacacttgcaaatggagagattcgcaaacgccctctaatcgaaactaatggggaaactggagaaattgtctgggataaagggcgagattttgccacagtgcgcaaagtattgtccatgccccaagtcaatattgtcaagaaaacagaagtacagacaggcggattctccaaggagtcaattttaccaaaaagaaattcggacaagcttattgctcgtaaaaaagactgggatccaaaaaaatatggtggttttgatagtccaacggtagcttattcagtcctagtggttgctaaggtggaaaaagggaaatcgaagaagttaaaatccgttaaagagttactagggatcacaattatggaaagaagttcctttgaaaaaaatccgattgactttttagaagctaaaggatataaggaagttaaaaaagacttaatcattaaactacctaaatatagtctttttgagttagaaaacggtcgtaaacggatgctggctagtgccggagaattacaaaaaggaaatgagctggctctgccaagcaaatatgtgaattttttatatttagctagtcattatgaaaagttgaagggtagtccagaagataacgaacaaaaacaattgtttgtggagcagcataagcattatttagatgagattattgagcaaatcagtgaattttctaagcgtgttattttagcagatgccaatttagataaagttcttagtgcatataacaaacatagagacaaaccaatacgtgaacaagcagaaaatattattcatttatttacgttgacgaatcttggagctcccgctgcttttaaatattttgatacaacaattgatcgtaaacgatatacgtctacaaaagaagttttagatgccactcttatccatcaatccatcactggtctttatgaaacacgcattgatttgagtcagctaggaggtgacggttctcccaagaagaagaggaaagtctcgagcggtggagctgcaggatgag

SEQ ID NO: 32 - DNA, 105 bp, bean yellow dwarf virus
atatgtaatcaaggacttgtttagagtttctagctggctggatattagggtgatttccttcaaaatcgaaaaaagaaggatccctaatacaaggttttttatcaa

SEQ ID NO: 33 - DNA, 102 bp, bean yellow dwarf virus
ggtagtgccatcttgatgaagctcagaagcaacaccaaggaagaaaataagaaaaggtgtgagtttctcccagagaaactgaataaatcatctctttgagat

SEQ ID NO: 34 - DNA, 4363 bp, Artificial Sequence
  construct including the Cas9 coding sequence flanked by both the left and right homology arms, operatively linked to the RNA polymerase II promoter
atatgtaatcaaggacttgtttagagtttctagctggctggatattagggtgatttccttcaaaatcgaaaaaagaaggatccctaatacaaggttttttatcaaatggataagaaatactcaataggcttagatatcggcacaaatagcgtcggatgggcggtgatcactgatgaatataaggttccgtctaaaaagttcaaggttctgggaaatacagaccgccacagtatcaaaaaaaatcttataggggctcttttatttgacagtggagagacagcggaagcgactcgtctcaaacggacagctcgtagaaggtatacacgtcggaagaatcgtatttgttatctacaggagattttttcaaatgagatggcgaaagtagatgatagtttctttcatcgacttgaagagtcttttttggtggaagaagacaagaagcatgaacgtcatcctatttttggaaatatagtagatgaagttgcttatcatgagaaatatccaactatctatcatctgcgaaaaaaattggtagattctactgataaagcggatttgcgcttaatctatttggccttagcgcatatgattaagtttcgtggtcattttttgattgagggagatttaaatcctgataatagtgatgtggacaaactatttatccagttggtacaaacctacaatcaattatttgaagaaaaccctattaacgcaagtggagtagatgctaaagcgattctttctgcacgattgagtaaatcaagacgattagaaaatctcattgctcagctccccggtgagaagaaaaatggcttatttgggaatctcattgctttgtcattgggtttgacccctaattttaaatcaaattttgatttggcagaagatgctaaattacagctttcaaaagatacttacgatgatgatttagataatttattggcgcaaattggagatcaatatgctgatttgtttttggcagctaagaatttatcagatgctattttactttcagatatcctaagagtaaatactgaaataactaaggctcccctatcagcttcaatgattaaacgctacgatgaacatcatcaagacttgactcttttaaaagctttagttcgacaacaacttccagaaaagtataaagaaatcttttttgatcaatcaaaaaacggatatgcaggttatattgatgggggagctagccaagaagaattttataaatttatcaaaccaattttagaaaaaatggatggtactgaggaattattggtgaaactaaatcgtgaagatttgctgcgcaagcaacggacctttgacaacggctctattccccatcaaattcacttgggtgagctgcatgctattttgagaagacaagaagacttttatccatttttaaaagacaatcgtgagaagattgaaaaaatcttgacttttcgaattccttattatgttggtccattggcgcgtggcaatagtcgttttgcatggatgactcggaagtctgaagaaacaattaccccatggaattttgaagaagttgtcgataaaggtgcttcagctcaatcatttattgaacgcatgacaaactttgataaaaatcttccaaatgaaaaagtactaccaaaacatagtttgctttatgagtattttacggtttataacgaattgacaaaggtcaaatatgttactgaaggaatgcgaaaaccagcatttctttcaggtgaacagaagaaagccattgttgatttactcttcaaaacaaatcgaaaagtaaccgttaagcaattaaaagaagattatttcaaaaaaatagaatgttttgatagtgttgaaatttcaggagttgaagatagatttaatgcttcattaggtacctaccatgatttgctaaaaattattaaagataaagattttttggataatgaagaaaatgaagatatcttagaggatattgttttaacattgaccttatttgaagatagggagatgattgaggaaagacttaaaacatatgctcacctctttgatgataaggtgatgaaacagcttaaacgtcgccgttatactggttggggacgtttgtctcgaaaattgattaatggtattagggataagcaatctggcaaaacaatattagattttttgaaatcagatggttttgccaatcgcaattttatgcagctgatccatgatgatagtttgacatttaaagaagacattcaaaaagcacaagtgtctggacaaggcgatagtttacatgaacatattgcaaatttagctggtagccctgctattaaaaaaggtattttacagactgtaaaagttgttgatgaattggtcaaagtaatggggcggcataagccagaaaatatcgttattgaaatggcacgtgaaaatcagacaactcaaaagggccagaaaaattcgcgagagcgtatgaaacgaatcgaagaaggtatcaaagaattaggaagtcagattcttaaagagcatcctgttgaaaatactcaattgcaaaatgaaaagctctatctctattatctccaaaatggaagagacatgtatgtggaccaagaattagatattaatcgtttaagtgattatgatgtcgatcacattgttccacaaagtttccttaaagacgattcaatagacaataaggtcttaacgcgttctgataaaaatcgtggtaaatcggataacgttccaagtgaagaagtagtcaaaaagatgaaaaactattggagacaacttctaaacgccaagttaatcactcaacgtaagtttgataatttaacgaaagctgaacgtggaggtttgagtgaacttgataaagctggttttatcaaacgccaattggttgaaactcgccaaatcactaagcatgtggcacaaattttggatagtcgcatgaatactaaatacgatgaaaatgataaacttattcgagaggttaaagtgattaccttaaaatctaaattagtttctgacttccgaaaagatttccaattctataaagtacgtgagattaacaattaccatcatgcccatgatgcgtatctaaatgccgtcgttggaactgctttgattaagaaatatccaaaacttgaatcggagtttgtctatggtgattataaagtttatgatgttcgtaaaatgattgctaagtctgagcaagaaataggcaaagcaaccgcaaaatatttcttttactctaatatcatgaacttcttcaaaacagaaattacacttgcaaatggagagattcgcaaacgccctctaatcgaaactaatggggaaactggagaaattgtctgggataaagggcgagattttgccacagtgcgcaaagtattgtccatgccccaagtcaatattgtcaagaaaacagaagtacagacaggcggattctccaaggagtcaattttaccaaaaagaaattcggacaagcttattgctcgtaaaaaagactgggatccaaaaaaatatggtggttttgatagtccaacggtagcttattcagtcctagtggttgctaaggtggaaaaagggaaatcgaagaagttaaaatccgttaaagagttactagggatcacaattatggaaagaagttcctttgaaaaaaatccgattgactttttagaagctaaaggatataaggaagttaaaaaagacttaatcattaaactacctaaatatagtctttttgagttagaaaacggtcgtaaacggatgctggctagtgccggagaattacaaaaaggaaatgagctggctctgccaagcaaatatgtgaattttttatatttagctagtcattatgaaaagttgaagggtagtccagaagataacgaacaaaaacaattgtttgtggagcagcataagcattatttagatgagattattgagcaaatcagtgaattttctaagcgtgttattttagcagatgccaatttagataaagttcttagtgcatataacaaacatagagacaaaccaatacgtgaacaagcagaaaatattattcatttatttacgttgacgaatcttggagctcccgctgcttttaaatattttgatacaacaattgatcgtaaacgatatacgtctacaaaagaagttttagatgccactcttatccatcaatccatcactggtctttatgaaacacgcattgatttgagtcagctaggaggtgacggttctcccaagaagaagaggaaagtctcgagcggtggagctgcaggatgagggtagtgccatcttgatgaagctcagaagcaacaccaaggaagaaaataagaaaaggtgtgagtttctcccagagaaactgaataaatcatctctttgagat

SEQ ID NO: 35 - DNA, 40 bp, Artificial Sequence
  crRNA comprising SEQ ID NO 15 as target sequence
aatttctactcttgtagatctggagaagagcatgatagtg

SEQ ID NO: 36 - DNA, 87 bp, Streptococcus pyogenes
gttggaaccattcaaaacagcatagcaagttaaaataaggctagtccgttatcaacttgaaaaagtggcaccgagtcggtgcttttt

(19) United States
(12) Patent Application Publication
(10) Pub. No.: US 2019/0352653 A1
(43) Pub. Date: Nov. 21, 2019

(54) CONFERRING RESISTANCE TO GEMINIVIRUSES IN PLANTS IN ALTERNATIVE MANNER TO GENE DRIVE, USING CRISPR/CAS SYSTEMS

(71) Applicant: KWS SAAT SE, Einbeck (DE)

(72) Inventors: Aaron Hummel, St. Louis, MO (US); Derek Graham Bartlem, St. Louis, MO (US)

(73) Assignee: KWS SAAT SE, Einbeck (DE)

(21) Appl. No.: 16/471,465

(22) PCT Filed: Dec. 20, 2017
(86) PCT No.: PCT/EP2017/083952

§ 371 Date: Jun. 19, 2019

Related U.S. Application Data
(60) Provisional application No. 62/436,792, filed on Dec. 20, 2016.

(51) Int. Cl. C12N 15/82

(52) U.S. Cl. CPC C12N 15/8213; C12N 2310/20; C12N 15/8283

(57) ABSTRACT

Materials and methods for conferring geminivirus resistance to plants or plant cells, and particularly to materials and methods for using CRISPR/Cas or CRISPR/Cpf1 systems to confer resistance to geminiviruses. Materials and methods are described to insert sequence at a double stranded break in a geminivirus genome.

### TECHNICAL FIELD

Disclosed are materials and methods for conferring geminivirus resistance to plants, and particularly to materials and methods for using site-specific nuclease (SDN) systems to confer resistance to geminiviruses to plants.

### BACKGROUND

Geminiviruses are a major plant pathogen, responsible for significant crop losses worldwide (Mansoor et al. Trends Plant Sci. 8:128-134, 2003). Geminiviruses are small, single-stranded, circular DNA plant viruses in a particle composed of two partially assembled icosahedra joined together. The size of the geminivirus genome is between 2.5 and 3.0 kb.

Due to a large host range, geminiviruses are capable of causing disease in a widevariety of plants, including species from both monocotyledonous (e.g., maize and wheat) and dicotyledonous (e.g., tomato and cassava) groups. Examples of geminiviruses include the cabbage leaf curl virus, tomato golden mosaic virus, bean yellow dwarf virus, African cassava mosaic virus, wheat dwarf virus, miscanthus streak mastrevirus, tobacco yellow dwarf virus, tomato yellow leaf curl virus, bean golden mosaic virus, beet curly top virus, maize streak virus, and tomato pseudo-curly top virus.

There is a need to provide resistance to geminivirus in plants through biologically and ecologically safe strategies, i.e. while at the same time minimizing release of any genetically modified geminivirus into the wild through a “gene drive” effect.

### SUMMARY OF THE DISCLOSURE

The present disclosure is based in part on the discovery of effective genome-engineering methods for increasing plant resistance to geminiviruses. The methods provided herein utilize the prokaryotic adaptive immune system known as the Clustered Regularly Interspaced Short Palindromic Repeats (CRISPR)/CRISPR-associated (Cas) system, which includes a nuclease known as Cas9. As an alternative, in any aspects and embodiments described herein, Cpf1 and NgAgo can be used instead of Cas9.

In one aspect is a method for generating a plant cell having an increased resistance to a geminivirus infection. The method comprises introducing into the genome of the plant cell:

(i) a first nucleic acid comprising a sequence encoding a site-specific nuclease protein, where the protein may be a CRISPR (system)-associated site-specific nuclease (SDN) protein, preferably a Cas protein for instance Cas9 or Cas10, or the Cpf1 protein, and where the protein is capable of introducing a double stranded break in the genome of the geminivirus at a target site, and where the first nucleic acid further comprises a promoter directing expression of the protein in the plant cell, or the expression of the protein is directed by the promoter of (ii) or (iii),

(ii) a second nucleic acid comprising a sequence encoding one or more CRISPR RNA (crRNA), where each of the one or more crRNA comprises a sequence complementary to one or more target sequences within the geminivirus genome, and where the second nucleic acid further comprises one or more promoters directing expression of the one or more crRNA in the plant cell, or the expression of the one or more crRNA is directed by the promoter of (i) or (iii) and

(iii) a third nucleic acid comprising a sequence encoding one or more trans-activating crRNA (tracrRNA), where the third nucleic acid further comprises one or more promoters directing expression of the one or more tracrRNA in the plant cell, or the expression of the one or more tracrRNA is directed by the promoter of (i) or (ii).

The second and third nucleic acids can be optionally replaced by a single fourth nucleic acid encoding one or more single guide RNA (sgRNA). Each of the one or more sgRNA comprises a sequence complementary to one or more target sequences within the geminivirus genome. The fourth nucleic acid further comprises one or more promoters directing expression of the one or more sgRNA in the plant cell, or the expression of the one or more sgRNA are directed by the promoter of (i). In case the site-specific nuclease protein is NgAgo the second and third nucleic acids can be replaced by a single fourth nucleic acid encoding one or more DNA molecules. Each of the one or more DNA molecules comprises a sequence complementary to one or more target sequences within the geminivirus genome and one or more DNA molecules are able to guide the NgAgo to the one or more target sequences within the geminivirus genome. The fourth nucleic acid further comprises one or more promoters directing expression of the one or more DNA molecules in the plant cell, or the expression of the one or more DNA molecules is directed by the promoter of (i).

The first and/or second and/or third nucleic acid or said first and/or fourth nucleic acid further comprises a left homology arm sequence and/or a right homology arm sequence. Each homology arm sequence comprises a nucleotide sequence from the geminivirus genome that is adjacent to the target site of the double stranded break introduced by the site-specific nuclease protein within a geminivirus target sequence. The left homology arm sequence and the right homology arm sequence are effective to introduce the nucleic acid sequences contained between them into the geminivirus genome at a geminivirus target sequence by homologous recombination. The first, second and third nucleic acid or said first and fourth nucleic acid become stably integrated into the genome of the plant cell.

In another aspect is a method for generating a plant cell having an increased resistance to a geminivirus infection. The method comprises introducing into the genome of the plant cell:

(i) a first nucleic acid sequence encoding a site-specific nuclease protein, where the protein may be a CRISPR (system)-associated site-specific nuclease protein, preferably a Cas protein, for instance Cas9 or Cas10, or the Cpf1 protein, where the protein is capable of introducing a double stranded break in the genome of the geminivirus at a target site, and where the first nucleic acid sequence is operably linked to a promoter directing expression of the protein in the plant cell, or where the first nucleic acid sequence is operably linked to the promoter of (ii) or (iii),

(ii) a second nucleic acid sequence encoding a CRISPR RNA (crRNA), where the crRNA comprises a sequence complementary to one or more target sequences within the geminivirus genome, and where the second nucleic acid sequence is operably linked to a promoter directing expression of the crRNA in the plant cell, or the second nucleic acid sequence is operably linked to the promoter of (i) or (iii), and

(iii) a third nucleic acid sequence encoding a trans-activating crRNA (tracrRNA), where the third nucleic acid sequence is operably linked to a promoter directing expression of the tracrRNA in the plant cell, or the third nucleic acid sequence is operably linked to the promoter of (i) or (ii).

The second and third nucleic acid sequences can be optionally replaced by a single fourth nucleic acid sequence encoding a hybrid crRNA/tracrRNA as a guide RNA (gRNA). The gRNA comprises a sequence complementary to a target sequence within the geminivirus genome, where the fourth nucleic acid sequence is operably linked to a promoter directing expression of the gRNA in the plant cell, or the fourth nucleic acid sequence is operably linked to the promoter of (i). In case the site-specific nuclease protein is NgAgo the second and third nucleic acids can be replaced by a single fourth nucleic acid encoding one or more DNA molecules. Each of the one or more DNA molecules comprises a sequence complementary to one or more target sequences within the geminivirus genome and one or more DNA molecules are able to guide the NgAgo to the one or more target sequences within the geminivirus genome. The fourth nucleic acid further comprises one or more promoters directing expression of the one or more DNA molecules in the plant cell, or the expression of the one or more DNA molecules is directed by the promoter of (i).

The first and/or second and/or third nucleic acid or the first and/or fourth nucleic acid further comprises a left homology arm sequence and a right homology arm sequence, each homology arm sequence comprising a nucleotide sequence from the geminivirus genome that is adjacent to the site of the double stranded break introduced by the protein within a geminivirus target sequence. The left homology arm sequence and the right homology arm sequence are effective to introduce the nucleic acid sequences contained between them into the geminivirus genome at or near a geminivirus target sequence by homologous recombination. The first, second and third nucleic acid or the first and fourth nucleic acid become stably integrated into the genome of the plant cell.

In another aspect is a method for generating a plant cell having an increased resistance to a geminivirus infection. The method comprises introducing into the genome of the plant cell one nucleic acid molecule comprising:

(i) a first nucleic acid sequence encoding a site-specific nuclease protein, where the protein may be a CRISPR (system)-associated site-specific nuclease protein, preferably a Cas protein, for instance Cas9 or Cas10, or the Cpf1 protein, where the protein is capable of introducing a double stranded break in the genome of the geminivirus at a target site, and where the first nucleic acid sequence is operably linked to a promoter directing expression of the protein in the plant cell, or where the first nucleic acid sequence is operably linked to the promoter of (ii) or (iii),

(ii) a second nucleic acid sequence encoding a CRISPR RNA (crRNA), where the crRNA comprises a sequence complementary to one or more target sequences within the geminivirus genome, and where the second nucleic acid sequence is operably linked to a promoter directing expression of the crRNA in the plant cell, or the second nucleic acid sequence is operably linked to the promoter of (i) or (iii), and

(iii) a third nucleic acid sequence encoding a trans-activating crRNA (tracrRNA), where the third nucleic acid sequence is operably linked to a promoter directing expression of the tracrRNA in the plant cell, or the third nucleic acid sequence is operably linked to the promoter of (i) or (ii).

The second and third nucleic acid sequences can be optionally replaced by a single fourth nucleic acid sequence encoding a hybrid crRNA/tracrRNA as a guide RNA (gRNA). The gRNA comprises a sequence complementary to a target sequence within the geminivirus genome, where the fourth nucleic acid sequence is operably linked to a promoter directing expression of the gRNA in the plant cell, or the fourth nucleic acid sequence is operably linked to the promoter of (i). In case the site-specific nuclease protein is NgAgo the second and third nucleic acids can be replaced by a single fourth nucleic acid encoding one or more DNA molecules. Each of the one or more DNA molecules comprises a sequence complementary to one or more target sequences within the geminivirus genome and one or more DNA molecules are able to guide the NgAgo to the one or more target sequences within the geminivirus genome. The fourth nucleic acid further comprises one or more promoters directing expression of the one or more DNA molecules in the plant cell, or the expression of the one or more DNA molecules is directed by the promoter of (i).

The one nucleic acid molecule further comprises a left homology arm sequence and a right homology arm sequence, each homology arm sequence comprising a nucleotide sequence from the geminivirus genome that is adjacent to the site of the double stranded break introduced by the protein within a geminivirus target sequence. The left homology arm sequence and the right homology arm sequence are effective to introduce the nucleic acid sequences contained between them into the geminivirus genome at or near a geminivirus target sequence by homologous recombination. The one nucleic acid molecule becomes stably integrated into the genome of the plant cell.

The size of the nucleic acid sequence between the left homology arm sequence and the right homology arm sequence may be such that upon homologous recombination into the geminivirus genome it prevents such genome e.g. from being packaged into viral particles and/or the geminivirus target sequence is a sequence of the geminivirus genome essential for the infection of a plant cell.

Preferably, the first and second nucleic acid, the first and third nucleic acid, the second and third nucleic acids, the first, second and third nucleic acid or the first and fourth nucleic acid are placed on a single nucleic acid molecule for introducing into the genome of the plant cell.

Upon exposure of the plant cells generated by above described method(s) to a genminivirus,

(a) the site-specific nuclease protein can mediate a double stranded break in the geminivirus genome at or near the geminivirus target site, and

(b) one or more of the first and/or second and/or third or one or more of the first and/or fourth nucleic acids can be inserted into the geminivirus genome at the double stranded break by homologous recombination to form a modified geminivirus;

where the modified geminivirus is unable to undergo one or more of replication, packaging, transport from the plant cell, or infection of a plant cell.

The one or more crRNA or the one or more sgRNA may comprise a sequence complementary to one or more target sequences within the genomes of more than one geminivirus species or more than one strain of the same geminivirus species. Each homology arm sequence may comprise a nucleotide sequence that is adjacent to the target site of the double stranded break introduced by the protein within target sequence, and the left homology arm sequence and the right homology arm sequence may be effective to introduce the nucleic acid sequences contained between them into the genomes of more than one geminivirus species or more than one strain of the same geminivirus species by homologous recombination.

In another aspect is a method for generating a plant having an increased resistance to a geminivirus infection, where the method comprising
(I) generating a plant cell having an increased geminivirus resistance by one of the above described methods for generating a plant cell having an increased resistance to a geminivirus infection, and
(II) regenerating a plant from the plant cell of (I).

In another aspect is a plant cell generated by one of the above described methods for generating a plant cell having an increased resistance to a geminivirus infection, or a plant or plant part generated by the above described method for generating a plant having an increased resistance to a geminivirus infection.

In another aspect is a vector comprising:

(i) a first nucleic acid comprising a sequence encoding a site-specific nuclease protein, where the protein may be a CRISPR (system)-associated site-specific nuclease protein, preferably a Cas protein, for instance Cas9 or Cas10, or the Cpf1 protein, and where the protein is capable of introducing a double stranded break in the genome of a geminivirus, and where the first nucleic acid further comprises a promoter directing expression of the protein in a plant cell, or the expression of the protein is directed by the promoter of (ii) or (iii),

(ii) a second nucleic acid comprising a sequence encoding one or more Clustered Regularly Interspaced Short Palindromic Repeats (CRISPR) RNA (crRNA), where each of the one or more crRNA comprises a sequence complementary to one or more target sequences within the geminivirus genome and where the second nucleic acid further comprises one or more promoters directing expression of the one or more crRNA in a plant cell, or the expression of the one or more crRNA is directed by the promoter of (i) or (iii), and

(iii) a third nucleic acid comprising a sequence encoding one or more trans-activating crRNA (tracrRNA), where the third nucleic acid further comprises one or more promoters directing expression of the one or more tracrRNA in a plant cell, or the expression of the one or more tracrRNA is directed by the promoter of (i) or (ii), and

(iv) a left homology arm sequence and/or a right homology arm sequence, each homology arm sequence comprising a nucleotide sequence from the geminivirus genome that is adjacent to the target site of the double stranded break introduced by the protein within a geminivirus target sequence, and where the left homology arm sequence and the right homology arm sequence are effective to introduce the nucleic acid sequences contained between them into a geminivirus genome at a geminivirus target sequence by homologous recombination.

In another aspect is a vector comprising:

(i) a first nucleic acid comprising a sequence encoding a site-specific nuclease protein, where the protein may be a CRISPR (system)-associated site-specific nuclease protein, preferably a Cas protein, for instance Cas9 or Cas10, or the Cpf1 protein, and where the protein is capable of introducing a double stranded break in the genome of a geminivirus, and where the first nucleic acid further comprises a promoter directing expression of the protein in a plant cell, or the expression of the protein is directed by the promoter of (ii) or (iii),

(ii) a second nucleic acid encoding one or more single guide RNA (sgRNA), where each of the one or more sgRNA comprises a sequence complementary to one or more target sequences within the geminivirus genome and where the second nucleic acid further comprises one or more promoters directing expression of the one or more sgRNA in a plant cell, or the expression of the one or more sgRNA is directed by the promoter of (i), and

(iii) a left homology arm sequence and/or a right homology arm sequence, each homology arm sequence comprising a nucleotide sequence from the geminivirus genome that is adjacent to the target site of the double stranded break introduced by the protein within a geminivirus target sequence, and where the left homology arm sequence and the right homology arm sequence are effective to introduce the nucleic acid sequences contained between them into a geminivirus genome at a geminivirus target sequence by homologous recombination.

In another aspect is a vector comprising:

(i) a first nucleic acid comprising a sequence encoding a site-specific nuclease protein, where the protein may be a CRISPR (system)-associated site-specific nuclease protein, preferably a Cas protein, for instance Cas9 or Cas10, or the Cpf1 protein, and where the protein is capable of introducing a double stranded break in the genome of a geminivirus, and where the first nucleic acid sequence is operably linked to a promoter directing expression of the protein in a plant cell, or the first nucleic acid sequence is operably linked to the promoter of (ii) or (iii),

(ii) a second nucleic acid comprising a sequence encoding a Clustered Regularly Interspaced Short Palindromic Repeats (CRISPR) RNA (crRNA), where the crRNA comprises a sequence complementary to one or more target sequences within the geminivirus genome and where the second nucleic acid sequence is operably linked to a promoter directing expression of the crRNA in a plant cell, or the second nucleic acid sequence is operably linked to the promoter of (i) or (iii),

(iii) a third nucleic acid encoding a trans-activating crRNA (tracrRNA), where the third nucleic acid sequence is operably linked to a promoter directing expression of the tracrRNA in a plant cell, or the second nucleic acid sequence is operably linked to the promoter of (i) or (ii), and

(iv) a left homology arm sequence and/or a right homology arm sequence, each homology arm sequence comprising a nucleotide sequence from the geminivirus genome that is adjacent to the target site of the double stranded break introduced by the protein within a geminivirus target sequence, and where the left homology arm sequence and the right homology arm sequence are effective to introduce the nucleic acid sequences contained between them into a geminivirus genome at a geminivirus target sequence by homologous recombination.

In another aspect is a vector comprising:

(i) a first nucleic acid comprising a sequence encoding a site-specific nuclease protein, where the protein may be a CRISPR (system)-associated site-specific nuclease protein, preferably a Cas protein, for instance Cas9 or Cas10, or the Cpf1 protein, and where the protein is capable of introducing a double stranded break in the genome of a geminivirus, and where the first nucleic acid sequence is operably linked to a promoter directing expression of the protein in a plant cell, or the first nucleic acid sequence is operably linked to the promoter of (ii),

(ii) a second nucleic acid sequence encoding a hybrid crRNA/tracrRNA (gRNA), where the gRNA comprises a sequence complementary to a target sequence within the geminivirus genome and where the second nucleic acid sequence is operably linked to a promoter directing expression of the gRNA in a plant cell, or the second nucleic acid sequence is operably linked to the promoter of (i), and

(iii) a left homology arm sequence and/or a right homology arm sequence, each homology arm sequence comprising a nucleotide sequence from the geminivirus genome that is adjacent to the target site of the double stranded break introduced by the protein within a geminivirus target sequence, and where the left homology arm sequence and the right homology arm sequence are effective to introduce the nucleic acid sequences contained between them into a geminivirus genome at a geminivirus target sequence by homologous recombination.

The size of the nucleic acid sequence between the left homology arm sequence and the right homology arm sequence may be such that upon homologous recombination into the geminivirus genome it prevents such genome e.g. from being packaged into viral particles and/or the geminivirus target sequence is a sequence of the geminivirus genome essential for the infection of a plant cell.

Preferably, the first and second nucleic acid, the first and third nucleic acid, the second and third nucleic acids, the first, second and third nucleic acid or the first and fourth nucleic acid are placed on a single nucleic acid molecule for introducing into the genome of the plant cell.

The one or more crRNA or the one or more sgRNA may comprise a sequence complementary to one or more target sequences within the genomes of more than one geminivirus species or more than one strain of the same geminivirus species. Each homology arm sequence may comprise a nucleotide sequence that is adjacent to the target site of the double stranded break introduced by the protein within target sequence, and the left homology arm sequence and the right homology arm sequence may be effective to introduce the nucleic acid sequences contained between them into the genomes of more than one geminivirus species or more than one strain of the same geminivirus species by homologous recombination.

In another aspect is a plant, plant part, or plant cell that has increased resistance to geminivirus infection, comprising one of the above described vectors.

In another aspect is a plant, plant part, or plant cell that has increased resistance to geminivirus infection, comprising stably integrated into the genome:

(i) a first nucleic acid comprising a sequence encoding a site-specific nuclease protein, where the protein may be a CRISPR (system)-associated site-specific nuclease protein, preferably a Cas protein, for instance Cas9 or Cas10, or the Cpf1 protein, and where the protein is capable of introducing a double stranded break in the genome of a geminivirus, and where the first nucleic acid further comprises a promoter directing expression of the protein in a plant cell, or the expression of the protein is directed by the promoter of (ii) or (iii),

(ii) a second nucleic acid comprising a sequence encoding one or more Clustered Regularly Interspaced Short Palindromic Repeats (CRISPR) RNA (crRNA), where each of the one or more crRNA comprises a sequence complementary to one or more target sequences within the geminivirus genome and where the second nucleic acid further comprises one or more promoters directing expression of the one or more crRNA in at least one plant cell, or the expression of the one or more crRNA is directed by the promoter of (i) or (iii), and

(iii) a third nucleic acid comprising a sequence encoding one or more trans-activating crRNA (tracrRNA), where the third nucleic acid further comprises one or more promoters directing expression of the one or more tracrRNA in at least one plant cell, or the expression of the one or more tracrRNA is directed by the promoter of (i) or (ii).

The second and third nucleic acids can be optionally replaced by a single fourth nucleic acid encoding one or more single guide RNA (sgRNA). Each of the one or more sgRNA comprises a sequence complementary to one or more target sequences within the geminivirus genome. The fourth nucleic acid further comprises one or more promoters directing expression of the one or more sgRNA in at least one plant cell, or the expression of the one or more sgRNA is directed by the promoter of (i).

The first and/or second and/or third nucleic acid or the first and/or fourth nucleic acid further comprises a left homology arm sequence and/or a right homology arm sequence. Each homology arm sequence comprises a nucleotide sequence from the geminivirus genome that is adjacent to the site of the double stranded break introduced by the protein within a geminivirus target sequence. The left homology arm sequence and the right homology arm sequence are effective to introduce the nucleic acid sequences contained between them into the geminivirus genome at a geminivirus target sequence by homologous recombination.

In another aspect is a plant, plant part, or plant cell that has increased resistance to geminivirus infection, comprising stably integrated into the genome:

(i) a first nucleic acid comprising a sequence encoding a site-specific nuclease protein, where the protein may be a CRISPR (system)-associated site-specific nuclease protein, preferably a Cas protein, for instance Cas9 or Cas10, or the Cpf1 protein, and where the protein is capable of introducing a double stranded break in the genome of a geminivirus, and, where the first nucleic acid sequence is operably linked to a promoter directing expression of the protein in at least one plant cell, or the first nucleic acid sequence is operably linked to the promoter of (ii) or (iii),

(ii) a second nucleic acid comprising a sequence encoding a Clustered Regularly Interspaced Short Palindromic Repeats (CRISPR) RNA (crRNA), where the crRNA comprises a sequence complementary to one or more target sequences within the geminivirus genome, and where the second nucleic acid sequence is operably linked to a promoter directing expression of the crRNA in at least one plant cell, or the second nucleic acid sequence is operably linked to the promoter of (i) or (iii), and

(iii) a third nucleic acid encoding a trans-activating crRNA (tracrRNA), where the third nucleic acid sequence is operably linked to a promoter directing expression of the tracrRNA in at least one plant cell, or the second nucleic acid sequence is operably linked to the promoter of (i) or (ii).

The second and third nucleic acid sequences can be optionally replaced by a single fourth nucleic acid sequence encoding a hybrid crRNA/tracrRNA (gRNA). The gRNA comprises a sequence complementary to a target sequence within the geminivirus genome. The fourth nucleic acid sequence is operably linked to a promoter directing expression of the gRNA in at least one plant cell, or the fourth nucleic acid sequence is operably linked to the promoter of (i).

The first and/or second and/or third nucleic acid or the first and/or fourth nucleic acid further comprises a left homology arm sequence and a right homology arm sequence. Each homology arm sequence comprises a nucleotide sequence from the geminivirus genome that is adjacent to the site of the double stranded break introduced by the protein within a geminivirus target sequence. The left homology arm sequence and the right homology arm sequence are effective to introduce the nucleic acid sequences contained between them into the geminivirus genome at or near a geminivirus target sequence by homologous recombination.

In another aspect is a plant, plant part, or plant cell that has increased resistance to geminivirus infection, comprising stably integrated into the genome one nucleic acid molecule comprising:

(i) a first nucleic acid comprising a sequence encoding a site-specific nuclease protein, where the protein may be a CRISPR (system)-associated site-specific nuclease protein, preferably a Cas protein, for instance Cas9 or Cas10, or the Cpf1 protein, and where the protein is capable of introducing a double stranded break in the genome of a geminivirus, and, where the first nucleic acid sequence is operably linked to a promoter directing expression of the protein in at least one plant cell, or the first nucleic acid sequence is operably linked to the promoter of (ii) or (iii),

(ii) a second nucleic acid comprising a sequence encoding a Clustered Regularly Interspaced Short Palindromic Repeats (CRISPR) RNA (crRNA), where the crRNA comprises a sequence complementary to one or more target sequences within the geminivirus genome, and where the second nucleic acid sequence is operably linked to a promoter directing expression of the crRNA in at least one plant cell, or the second nucleic acid sequence is operably linked to the promoter of (i) or (iii), and

(iii) a third nucleic acid encoding a trans-activating crRNA (tracrRNA), where the third nucleic acid sequence is operably linked to a promoter directing expression of the tracrRNA in at least one plant cell, or the second nucleic acid sequence is operably linked to the promoter of (i) or (ii).

The second and third nucleic acid sequences can be optionally replaced by a single fourth nucleic acid sequence encoding a hybrid crRNA/tracrRNA (gRNA). The gRNA comprises a sequence complementary to a target sequence within the geminivirus genome. The fourth nucleic acid sequence is operably linked to a promoter directing expression of the gRNA in at least one plant cell, or the fourth nucleic acid sequence is operably linked to the promoter of (i).

The one nucleic acid molecule further comprises a left homology arm sequence and a right homology arm sequence. Each homology arm sequence comprises a nucleotide sequence from the geminivirus genome that is adjacent to the site of the double stranded break introduced by the protein within a geminivirus target sequence. The left homology arm sequence and the right homology arm sequence are effective to introduce the nucleic acid sequences contained between them into the geminivirus genome at or near a geminivirus target sequence by homologous recombination.

The size of the nucleic acid sequence between the left homology arm sequence and the right homology arm sequence may be such that upon homologous recombination into the geminivirus genome it prevents such genome e.g. from being packaged into viral particles and/or the geminivirus target sequence is a sequence of the geminivirus genome essential for the infection of a plant cell.

Preferably, the first and second nucleic acid, the first and third nucleic acid, the second and third nucleic acids, the first, second and third nucleic acid or the first and fourth nucleic acid are placed on a single nucleic acid molecule for introducing into the genome of the plant cell.

Upon exposure of the plant cells generated by above described method(s) to a genminivirus,

(a) the site-specific nuclease protein can mediate a double stranded break in the geminivirus genome at or near the geminivirus target site, and

(b) one or more of the first and/or second and/or third or one or more of the first and/or fourth nucleic acids can be inserted into the geminivirus genome at the double stranded break by homologous recombination to form a modified geminivirus;

where the modified geminivirus is unable to undergo one or more of replication, packaging, transport from the plant cell, or infection of a plant cell.

The one or more crRNA or the one or more sgRNA may comprise a sequence complementary to one or more target sequences within the genomes of more than one geminivirus species or more than one strain of the same geminivirus species. Each homology arm sequence may comprise a nucleotide sequence that is adjacent to the target site of the double stranded break introduced by the protein within target sequence, and the left homology arm sequence and the right homology arm sequence may be effective to introduce the nucleic acid sequences contained between them into the genomes of more than one geminivirus species or more than one strain of the same geminivirus species by homologous recombination.

The details of one or more embodiments of the invention are set forth in the accompanying drawings and the description below. Other features, objects, and advantages of the invention will be apparent from the description and drawings, and from the claims.

### BRIEF DESCRIPTION OF THE DRAWINGS

FIG. 1 illustrates three nucleic acids to be integrated into a plant genome for instance by plant transformation: a Cas9 coding sequence flanked by a left and right homology arms to effect homologous recombination into a geminivirus genome, a crRNA, and a tracrRNA.

FIG. 2 illustrates a double stranded break in geminivirus genome from Cas9 protein, followed by insertion of Cas9 coding sequence into the geminivirus genome by way of homologous recombination.

FIG. 3A illustrates packaging of a geminivirus genome into a capsid.

FIG. 3B illustrates failure to package a geminivirus genome with Cas9 inserted due to excess size of the modified geminivirus genome.

FIG. 4 illustrates two nucleic acids to be integrated into a plant genome by plant transformation: a single nucleic acid with Cas9 coding sequence, a crRNA and tracrRNA flanked by left and right homology arms to effect homologous recombination into a geminivirus genome.

FIG. 5 illustrates a single nucleic acid with Cas9 coding sequence, crRNA and tracrRNA flanked by left and right homology arms to effect homologous recombination.

FIG. 6 illustrates two nucleic acids to be integrated into a plant genome by plant transformation: a single nucleic acid with Cas9 coding sequence flanked by left and right homology arms to effect homologous recombination into a geminivirus genome, and a crRNA and tracrRNA flanked by left and right homology arms to effect homologous recombination into a geminivirus genome.

### DETAILED DESCRIPTION

The methods described herein can be used for engineering plants with pre-programmed systems for gene editing like CRISPR/Cas or CRISPR/Cpf1 that target and disrupt geminivirus DNA sequences, and add additional sequence to the geminivirus genome, so as to inhibit packaging, movement or transport of the modified geminivirus from an infected cell, or infection of additional plant cells. It has the added benefit of amplifying the copy number of the gene that is recombined into the geminivirus genome, thereby increasing the expression potential for this component of the nuclease during the infection. For example CRISPR systems are used to generate double strand breaks (DSBs) in the target region. Disruption of the geminivirus with a DSB on its own can lead to increased plant resistance to geminivirus.

CRISPR systems in the native context provide bacteria and archaea with immunity to invading foreign nucleic acids and relies on RNA base pairing to direct an enzyme, such as Cas9 or Cpf1, to cleave DNA or RNA. Beside of CRISPR systems the NGAgo-based system relies on DNA base pairing to direct an Argonaute enzyme to cleave DNA or RNA. CRISPR systems are based on (a) small RNAs that base-pair with sequences carried by invading nucleic acid, and (b) a specialized class of endonucleases that cleave nucleic acids complementary to the small RNA. The CRISPR system can be reprogrammed to create targeted DSBs in DNA of higher-eukaryotic genomes, including animal and plant cells (Mali et al., Science 339:823-826, 2013; and Li et al., Nature Biotechnology 31(8): 688-691, 2013). Double stranded DNA virus genomes can be targeted by CRISPR system.

In addition to creating DSBs, CRISPR system can be further used for gene editing by triggering DNA repair pathways effective to introduce DNA at the site of the double stranded break. DNA can be added by homology-directed repair (HDR), involving homologous recombination between (1) the geminivirus genome and (2) sequence substantially identical to the genome near the DSB. The substantially identical sequence is both 5′ and 3′ to sequence desired to be inserted, and is collectively referred to as left and right homology arms. For example, if a DNA segment comprising a left homology arm, DNA to be inserted, and a right homology arm undergoes a crossover event in each of the left and right arms with geminivirus genomic DNA, the sequence between the homology arms can be inserted into the genome.

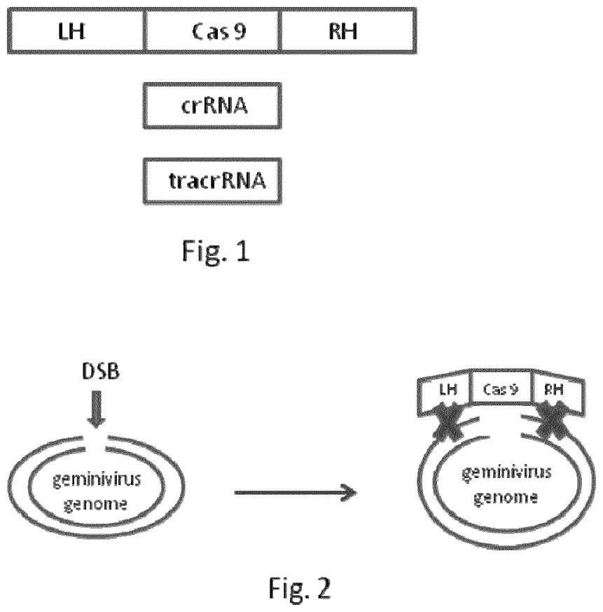

FIG. 1 shows exemplary constructs with three CRISPR/site-directed nuclease (SDN) reagents that can target a virus genome: Cas9, crRNA, and tracrRNA. LH refers to the left homology arm and RH refers to the right homology arm. By having SDN flanked by LH and RH that overlap with geminivirus genomic DNA around a DSB, SDN can be inserted into the genomic DNA by homologous recombination, which involves a double crossing over illustrated in FIG. 2. Cas9 can be replaced by any other site-directed nuclease, e.g. Cpf1.

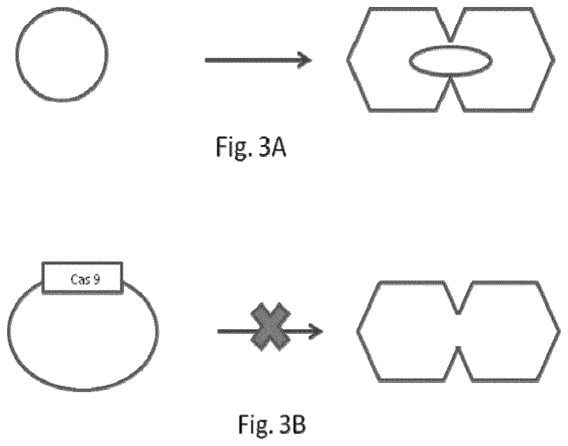

Further beneficial effects can occur by adding sequence into the geminivirus genome at the DSB. Inserting sequence into the geminivirus at the DSB can accelerate and amplify the process of preventing geminivirus replication and packaging in an infected plant cell as well as preventing geminivirus with modified sequence from escaping the cell or infecting a new cell. A geminivirus having added sequence may be too large to be effectively packaged into a capsid or otherwise transported out of the cell. FIG. 3A shows packaging of a wildtype geminivirus genome into a capsid having a double hexagonal shape. In FIG. 3B, geminivirus genome with an added Cas9 sequence is too large to be packaged into such capsid. Thus, the various aspects and embodiments described herein that add sequence to the genome solve a problem of providing increased geminivirus resistance without using a “gene drive” or otherwise releasing into the wild any amount of geminivirus with modified genomes.

Some of the terms used in this application will now be explained in more detail:

Unless stated otherwise, a “plant” may be any species of dicotyledon, monocotyledon or gymnosperm plant. The plants may be monocotyledon and of interest in agriculture or horticulture or for the production of bioenergy (bioethanol, biogas, etc). Examples are barley (Hordeum vulgare), sorghum (Sorghum bicolor), rye (Secale cereale), Triticale, sugar cane (Saccharum officinarium), maize (Zea mays), foxtail millet (Setaria italic), rice (Oryza sativa), Oryza minuta, Oryza australiensis, Oryza alta, wheat (Triticum aestivum), Triticum durum, Hordeum bulbosum, purple false brome (Brachypodium distachyon), sea barley (Hordeum marinum), goat grass (Aegilops tauschii), apple (Malus domestica), Beta vulgaris, sunflower (Helianthus annuus), Australian carrot (Daucus glochidiatus), American wild carrot (Daucus pusillus), Daucus muricatus, carrot (Daucus carota), eucalyptus (Eucalyptus grandis), Erythranthe guttata, Genlisea aurea, woodland tobacco (Nicotiana sylvestris), tobacco (Nicotiana tabacum), Nicotiana tomentosiformis, tomato (Solanum lycopersicum), potato (Solanum tuberosum), coffee (Coffea canephora), grape vine (Vitis vinifera), cucumber (Cucumis sativus), mulberry (Morus notabilis), thale cress (Arabidopsis thaliana), Arabidopsis lyrata, sand rock-cress (Arabidopsis arenosa), Crucihimalaya himalaica, Crucihimalaya wallichii, wavy bittercress (Cardamine flexuosa), peppergrass (Lepidium virginicum), sheperd's-purse (Capsella bursa-pastoris), Olmarabidopsis pumila, hairy rockcress (Arabis hirsuta), rape (Brassica napus), broccoli (Brassica oleracea), Brassica rapa, Brassica juncacea, black mustard (Brassica nigra), radish (Raphanus sativus), Eruca vesicaria sativa, orange (Citrus sinensis), Jatropha curcas, Gossypium sp., Musa sp., Glycine max, black cottonwood (Populus trichocarpa), Avena sp., turf grass and forage grass. The plant may be from the genus Zea, in particular the species Zea mays, or from the genus Beta, in particular the species Beta vulgaris.

Plant “parts” are in particular plant cells, plant tissue, in particular plant propagation material, preferably leaves, stems, roots, emerged radicles, flowers or flower parts, petals, fruits, pollen, pollen tubes, anther filaments, ovules, embryo sacs, egg cells, ovaries, zygotes, embryos, zygotic embryos per se, somatic embryos, hypocotyl sections, apical meristems, vascular bundles, pericycles, seeds, roots, cuttings, cell or tissue cultures, or any other part or product of a plant. The term “plant cell” should be understood to refer to isolated plant cells with a cell wall or aggregates thereof or protoplasts, for example.

The plant cell can be from a plant selected from the group consisting of barley (Hordeum vulgare), sorghum (Sorghum bicolor), rye (Secale cereale), Triticale, sugar cane (Saccharum officinarium), maize (Zea mays), foxtail millet (Setaria italic), rice (Oryza sativa), Oryza minuta, Oryza australiensis, Oryza alta, wheat (Triticum aestivum), Triticum durum, Hordeum bulbosum, purple false brome (Brachypodium distachyon), sea barley (Hordeum marinum), goat grass (Aegilops tauschii), apple (Malus domestica), Beta vulgaris, sunflower (Helianthus annuus), Australian carrot (Daucus glochidiatus), American wild carrot (Daucus pusillus), Daucus muricatus, carrot (Daucus carota), eucalyptus (Eucalyptus grandis), Erythranthe guttata, Genlisea aurea, woodland tobacco (Nicotiana sylvestris), tobacco (Nicotiana tabacum), Nicotiana tomentosiformis, tomato (Solanum lycopersicum), potato (Solanum tuberosum), coffee (Coffea canephora), grape vine (Vitis vinifera), cucumber (Cucumis sativus), mulberry (Morus notabilis), thale cress (Arabidopsis thaliana), Arabidopsis lyrata, sand rock-cress (Arabidopsis arenosa), Crucihimalaya himalaica, Crucihimalaya wallichii, wavy bittercress (Cardamine flexuosa), peppergrass (Lepidium virginicum), sheperd's-purse (Capsella bursa-pastoris), Olmarabidopsis pumila, hairy rockcress (Arabis hirsuta), rape (Brassica napus), broccoli (Brassica oleracea), Brassica rapa, Brassica juncacea, black mustard (Brassica nigra), radish (Raphanus sativus), Eruca vesicaria sativa, orange (Citrus sinensis), Jatropha curcas, Gossypium sp., Musa sp., Glycine max, black cottonwood (Populus trichocarpa), Avena sp., turf grass and forage grass. After the plant cell is transformed, it may later be introduced into its corresponding native plant. The methods provided herein can be useful for any type of crop or economically valuable plant that is susceptible to geminivirus infection and is amenable to stable DNA integration.

“Integration” into the genome of a plant of the previous parental generation allows the polynucleotide to be further inherited in a stable manner.

The expression “resistance” or “resistant” as regards a pathogen should be understood to mean the ability of a plant or plant cell to resist the damaging effects of the pathogen and extends from a delay in the development of disease to complete suppression of the development of the disease.

The term “increased resistance,” as used herein, means that a plant, plant part, or plant cell is less severely affected by geminivirus infection than a corresponding plant, plant part, or plant cell that does not contain any of the gene editing components like CRISPR/Cas or CRISPR/Cpf1 or nucleic acids as described herein. For example, a plant with increased resistance to geminivirus will display fewer or milder symptoms, e.g., leaf curling, chlorotic lesions, yellowing and stunting, when exposed to geminivirus, as compared to a corresponding plant that does not have increased geminivirus resistance. In some cases, symptoms of geminivirus infection can be scored by using a scale with no observable symptoms at one end and severe symptoms at the other. In such cases, the difference between the score for a plant with increased geminivirus resistance and the score for no observable symptoms will be less than the difference between the score for a corresponding plant without increased geminivirus resistance and the score for no observable symptoms.

“Operatively linked” means linked in a common nucleic acid molecule in a manner such that the linked elements are positioned and orientated with respect to each other such that transcription of the nucleic acid molecule can take place. A DNA which is operatively linked with a promoter is under the transcriptional control of this promoter.

As used herein, a “vector” is a replicon, such as a plasmid, phage, or cosmid, into which one or multiple DNA segments may be inserted so as to bring about the replication of the inserted segment.

In various embodiments, the first and/or second and/or third and/or fourth nucleic acid further comprises a left homology arm sequence and/or a right homology arm sequence. Any one or more of the first, second, third and fourth nucleic acid sequences may be preceded by a promoter directing expression in the plant cell.

Without wishing to be bound by theory, repair of a DSB occurs generally through non-homologous end joining (NHEJ) or homology-directed repair (HDR). NHEJ can quickly seal the double stranded break while introducing mutations that result in frame-shift, missense or nonsense mutation. HDR, on the other hand, can introduce sequence at the site of the DSB through homologous recombination. In various embodiments described herein, HDR allows for nucleic acids such as Cas9 or Cpf1, crRNA and tracrRNA, to be introduced at the DSB. Adding these nucleic acid sequences into the geminivirus genome can have multiple beneficial effects. With either HDR- or NHEJ-mediated repair, the geminivirus genome can be disrupted in a manner sufficient to block replication, packaging or transport of the virus. Infection of neighboring cells can be controlled or stopped, which may lead to overall increased resistance of a plant to geminivirus.

In various embodiments, insertion of sequence into the geminivirus genome results in enlargement of the geminivirus genome such that it cannot be packaged or otherwise be transported from one plant cell to another. The wildtype geminivirus genome generally has one or two components, each between 2.5 to 3.2 kb in size. The genome size can be increased by 200 bp to 9 kb, or any value in between, so as to block replication. Increasing the size of the genome can prevent packaging of geminivirus into capsids, transport of the geminivirus genome from one cell to another, or both. It can therefore be beneficial to have plant cells capable of both blocking geminivirus transmission and spreading modified geminivirus into the wild.

In various embodiments, after insertion of CRISPR/Cas nucleic acids into the geminivirus genome, transcription and expression of such nucleic acids greatly increases. The replication machinery, promoters and gene expression drivers that are used to replicate and package geminivirus can provide for high levels of transcription and expression of CRISPR/Cas nucleic acids and proteins, particularly in a positive-feedback manner. Such increased expression can be helpful to control high copy number geminivirus that start replicating after infecting a cell. Use of such positive-feedback mechanisms can be less burdensome to plant cells than expressing similarly high levels of CRISPR/SDN components in the absence of geminivirus.

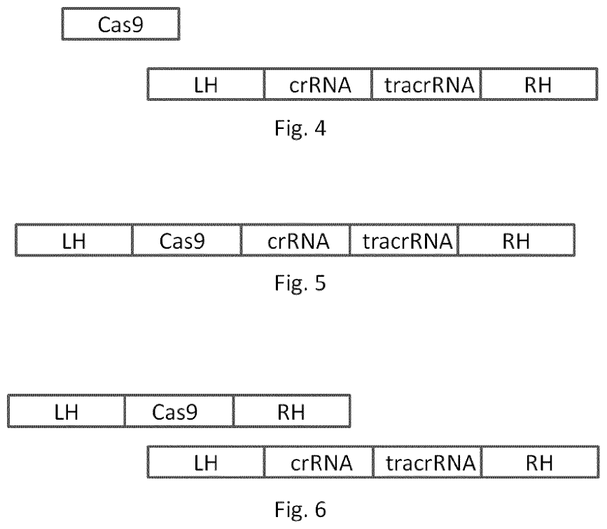

In various embodiments, the first, second, third and optionally fourth nucleic acids may be part of a vector, a single nucleic acid construct, or in separate constructs. Thus, in some cases it may be most efficient to include the sequences encoding the SDN protein, the crRNA(s), and the tracrRNA(s) in a single construct, e.g., a single vector. FIG. 5 shows an exemplary single construct having Cas9, crRNA and tracrRNA between left and right homology arms. In some embodiments, the crRNA and tracrRNA sequences can be present in separate nucleic acid constructs, e.g., separate vectors (see FIGS. 4 and 6).

For example, if a geminivirus is to be targeted at five different sequences, at least seven different nucleic acid constructs could be used for integration into the plant cell genome. A first nucleic acid encodes the SDN protein, a second nucleic acid encodes the tracrRNA, and third through seventh nucleic acids encode the crRNAs. The left and right homology arm sequences may be added into any of these vectors to allow particular sequences between the homology arms to be integrated into the geminivirus genome.

In the various aspects and embodiments described herein, introducing of nucleic acids into the genome of a plant can be undertaken by various ways, including Agrobacterium-mediated transformation, electroporation, polyethylene glycol (PEG), insect vectors, grafting, DNA abrasion, viral vectors, insertion by CRISPR systems followed by homologous recombination, and biolistic transformation. Exemplary viral vectors are from a DNA virus such as geminiviruses, nanoviruses (e.g., fava bean necrotic yellow virus), an RNA virus such as, a tobravirus (e.g., tobacco rattle virus or tobacco mosaic virus), a potexvirus (e.g., potato virus X), and a hordeivirus (e.g., barley stripe mosaic virus).

In the various aspects and embodiments described herein, the SDN protein is a Cas, preferably a Cas9 protein with nuclease activity. Other proteins with similar targeted nuclease activity can be used instead of Cas9, such as Cpf1, Cas10 and NgAgo. Cpf1 protein is capable of modifying DNA by introducing a double stranded break with 5′ overhangs, and may be used. NgAgo may also be used.

Since geminiviruses are circular, single-stranded (plus strand) DNA molecules that replicate through double-stranded DNA intermediates, the nuclease-active version of SDN can induce targeted double-strand breaks (DSBs) in the geminivirus double-stranded DNA replication intermediate. Also, wildtype SDN protein may be used to introduce such DSBs via its nuclease activity. Any mutants of SDN may be used that are capable of introducing DSBs, such as those that retain nuclease activity.

The crRNA and tracrRNA sequences can be engineered as a single crRNA/tracrRNA hybrid, also referred to herein as a “guide RNA” (gRNA).

Alternatively, in various embodiments the SDN protein can contain one or more mutations, e.g., substitutions, deletions, or additions, within its amino acid sequence as compared to the amino acid sequence of a corresponding wild type SDN protein, in which the mutant SDN retains nuclease activity. In some embodiments, additional amino acids may be added to the N- and/or C-termini. For example, Cas9 protein can be modified by adding a VP64 activation domain or a green fluorescent protein to the C-terminus, or by the adding nuclear-localization signals to both the N- and C-termini (see, e.g., Mali et al., Nature Biotechnology 31:833-838, 2013; and Cong et al., Science 339:819-823).

In various embodiments, the geminivirus homology regions within each crRNA sequence can be between about 10 and about 40 (e.g., 10, 11, 12, 13,14, 15, 16, 17, 18, 19, 20, 21, 22, 23, 24, 25, 26, 27, 28, 29, 30, 31, 32, 33, 34, 35, 36, 37, 38, 39, or 40) nucleotides in length. The tracrRNA hybridizing region within each crRNA sequence can be between about 8 and about 20 (e.g., 8, 9, 10, 11, 12, 13, 14, 15, 16, 17, 18, 19, or 20) nucleotides in length. The overall length of a crRNA sequence can be, for example, between about 20 and about 80 (e.g., 20, 25, 30, 35, 40, 45, 50, 55, 60, 65, 70, 75, or 80) nucleotides, while the overall length of a tracrRNA can be, for example, between about 10 and about 30 (e.g., 10, 12, 14, 16, 18, 20, 22, 24, 26, 28, or 30) nucleotides.

In various embodiments, multiple (e.g., one, two, three, four, five, or more than five) crRNA sequences, and multiple (e.g., one, two, three, four, five, or more than five) tracrRNA sequences can be included to allow crRNA sequences to be targeted to one or more (e.g., one, two, three, four, five, or more than five) geminivirus sequences. For example, each of the one or more crRNA sequences can contain a region that is homologous to a geminivirus sequence, such that the one or more crRNA sequences are targeted to different geminivirus sequences. The tracrRNA hybridizes with the crRNA, and together they guide the SDN protein to the target sequence. In some embodiments, when multiple crRNA sequences are used, each crRNA sequence can contain a different geminivirus homology region but the same tracrRNA hybridizing region. In addition multiple crRNA sequences can also be targeted to different molecules of the geminivirus DNA (e.g., to primary and satellite genomes).

In various embodiments, a gRNA is used instead of separate crRNA and tracrRNA sequences, with the gRNA sequence including a geminivirus homology region and a stem loop region that contains a crRNA/tracrRNA hybridizing region and a linker-loop sequence. The length of the gRNA can be between about 30 and about 130 (e.g., 30, 35, 40, 45, 50, 55, 60, 65, 70, 75, 80, 85, 90, 95, 100, 105, 110, 115, 120, 125, or 130) nucleotides.

The crRNA sequence is targeted to a sequence contained within a geminivirus genome. Suitable target sequences typically are followed by a protospacer adjacent motif (PAM) sequence that is required for cleavage. The PAM sequence can be immediately downstream of the target sequence. An NGG PAM sequence downstream of the target sequence can be required for cleavage by a Cas9 nuclease (e.g., by a S. pyogenes Cas9). Alternatively, a NNNNGMTT PAM sequence can be required for cleavage by a Neisseria meningitides Cas9 protein. The person skilled in the art knows that there are also other PAM requirements for different versions of Cas9. Furthermore, Cpf1 requires for instance a 5′ TTN or TTTN PAM.

In various embodiments, the target sequence is within a Rep protein ORF, a movement protein ORF, a coat protein ORF, a hairpin region that mediates replication origin, the origin of replication, the nuclear shuttling protein coding sequence, or a satellite DNA. Also, the target sequence may be the TAATATTAC sequence present in the apex of the geminivirus stem-loop structure. In various embodiments, a target sequence is found within one, or even more than one, of the following: tomato golden mosaic virus (TGMV), bean yellow dwarf virus (BeYDV), tomato yellow leaf curl virus (TYLCV), cabbage leaf curl virus, wheat dwarf virus, tomato leaf curl virus, maize streak virus, tobacco leaf curl virus, beet curly top virus, spinach severe curly top virus, bean golden mosaic virus, tomato pseudo-curly top virus, and turnip curly top virus.

The crRNA sequence can be targeted to a sequence within a geminivirus genome that is conserved across two or more species of geminivirus. Target sequences that are present in more than one geminivirus species, i.e. multiple geminivirus species, or present in more than one strain of the same geminivirus species, i.e. multiple strains of the same geminivirus species, can be useful to provide broader spectrum resistance to the plant. It is also possible to target multiple sequences with a geminivirus genome of a single species. This may have the benefit of increasing the potential for durability off resistance against that species. As an alternative, each of the one or more crRNA sequences can be fused to a tracrRNA sequence to form a guide RNA (gRNA). The tracrRNA sequence and the one or more crRNA sequences or the SDN sequence can be operably linked to a constitutive promoter (e.g., an RNA polymerase III promoter or an RNA polymerase II promoter), an inducible promoter (e.g. a promoter inducible in response to a geminivirus infection), or a plant tissue specific promoter.

The target sequence may comprise sequence from any of the following target sites:

A) origin of replication of TGMV:
(SEQ ID NO: 1)
GGTAGTAAGGTAG
B) bean yellow dwarf virus:
(SEQ ID NO: 2)
TGGAGGCATGGAGGCA
C) Rep coding sequence of BeYDV, Motif I:
(SEQ ID NO: 3)
TTCCTTACCTAT
D) Rep coding sequence of BeYDV, Motif II:
(SEQ ID NO: 4)
CACTATCATGCTCTTCTC
E)Rep coding sequence of BeYDV, Motif III:
(SEQ ID NO: 5)
GTCCTTGATTACATATCAAAG
F) TGMV, Motif I:
(SEQ ID NO: 6)
TTTCTTACATATCCTCAGTGC
G) TGMV, Motif II:
(SEQ ID NO: 7)
CACCTCCACGTGCTTATTCAG
H) TGMV, Motif III:
(SEQ ID NO: 8)
GTCAAGACGTACATCGACAAA
I) hairpin structure in ORF, BeYDV:
(SEQ ID NO: 9)
GCGACAAGGGGGGGCCCACGCCG
J) the nuclear shuttling protein (NSP) coding
sequence, cabbage leaf curl virus:
(SEQ ID NO: 10)
ATGTATCCTACAAAGTTTAGGCGTGG
K) movement protein(MP) coding sequence, cabbage
leaf curl virus:
(SEQ ID NO: 11)
CTATGTAATTAAACGCATTTGGAG

The geminivirus target sequence can be present on the plus or minus strand of the double stranded DNA intermediate form of the geminivirus genome. Examples of target sequence present on the plus strand include:

A) the conserved Rep binding sequence within the
origin of replication of TGMV:
(SEQ ID NO: 12)
GGTAGTAAGGTAG
B) the conserved Rep binding sequence within the
origin of replication of BeYDV-m:
(SEQ ID NO: 13)
TGGAGGCATGGAGGCA
C) Motif I within the Rep coding sequence of
BeYDV-m:
(SEQ ID NO: 14)
ATAGGTAAGGAA
D) Motif II within the Rep coding sequence of
BeYDV-m:
(SEQ ID NO: 15)
CTGGAGAAGAGCATGATAGTG
E) Motif III within the Rep coding sequence of
BeYDV-m:
(SEQ ID NO: 16)
CTTTGATATGTAATCAAGGAC,
F) Rb binding domain within the Rep coding
sequence of BeYDV-m:
(SEQ ID NO: 17)
TTCGTGGCAGCGAAG
G) the stem-loop structure within the origin of
replication of BeYDV-m:
(SEQ ID NO: 18)
GCGACAAGGGGGGGCCCACGCCG,
H) the nonanucleotide sequence present in the apex
of the stem-loop structure of BeYDV-m:
TAATATTAC,
I) the NSP coding sequence of the cabbage leaf
curl virus:
(SEQ ID NO: 19)
ATGTATCCTACAAAGTTTAGGCGTGG
J) the MP coding sequence of the cabbage leaf curl
virus:
(SEQ ID NO: 20)
CTATGTAATTAAACGCATTT.

Examples of target sequence present on the plus strand include:

A) the conserved Rep binding sequence within the
origin of replication of TGMV:
(SEQ ID NO: 21)
CTACCTTACTACC
B) the conserved Rep binding sequence within the
origin of replication of BeYDV-m:
(SEQ ID NO: 22)
TGCCTCCATGCCTCCA
C) Motif I within the Rep coding sequence of
BeYDV-m:
(SEQ ID NO: 23)
TTCCTTACCTAT
D) Motif II within the Rep coding sequence of
BeYDV-m:
(SEQ ID NO: 24)
CACTATCATGCTCTTCTCCAG
E) Motif III within the Rep coding sequence of
BeYDV-m:
(SEQ ID NO: 25)
GTCCTTGATTACATATCAAAG,
F) Rb binding domain within the Rep coding
sequence of BeYDV-m:
(SEQ ID NO: 26)
CTTCGCTGCCACGAA,
G) the stem-loop structure within the origin of
replication of BeYDV-m:
(SEQ ID NO: 27)
CGGCGTGGGCCCCCCCTTGTCGC,
H) the nonanucleotide sequence present in the apex
of the stem-loop structure of the BeYDV-m:
GTAATATTA
I) the NSP coding sequence of cabbage leaf curl
virus:
(SEQ ID NO: 28)
CCACGCCTAAACTTTGTAGGATACAT,
and
J) the MP coding sequence of cabbage leaf curl
virus:
(SEQ ID NO: 29)
AAATGCGTTTAATTACATAG

In various embodiments, the plant cell to be transformed by introducing one or more nucleic acids can be in a plant or in vitro. With respect to transforming such plant cells in various embodiments, the individual nucleic acid sequences, i.e. the first nucleic acid, the second nucleic acid, the third nucleic acid, and the optional fourth nucleic acid, are in a single vector or in separate vectors. In some embodiments, the fourth nucleic acid can encode a polycistronic message containing a tracrRNA sequence and one or more crRNA sequences, or containing a crRNA/tracrRNA hybrid (gRNA) sequence.

The introduction of the nucleic acid into the plant cell may allow the plant cell to express sufficient SDN protein, crRNA and tracrRNA such that when the plant cell is exposed to a geminivirus, the SDN protein, crRNA and tracrRNA work in concert to introduce a DSB at the target region in the geminivirus. On its own, the DSB can disrupt the replication, packaging, movement, transport of that geminivirus and reinfection of other plant cells.

Further, the plant cell may contain sufficient amounts of the nucleic acid sequence comprising the first, second and third nucleic acids such that sequences of the first, second and third nucleic acids are introduced at the DSB by homologous recombination. Introduction of such nucleic acid may further block packaging and transport due to inability to further package the enlarged genome into the capsid. See, FIG. 5.

In various embodiments, the left and right homology arms of nucleic acid sequence (iv) comprise nucleic acid sequence from the geminivirus genome that is adjacent to the geminivirus target site. In some embodiments, the left homology arm comprises nucleic acid sequence 5′ to the geminivirus target site and the right homology arm comprises nucleic acid sequence 3′ to the geminivirus target site. Alternatively, if the left homology arm comprises nucleic acid sequence 3′ to the geminivirus target site, then the right homology arm may comprise nucleic acid sequence 5′ to the geminivirus target site. The left homology arm and the right homology arm can comprise sequence that is within 10, 20, 30, 40, 50 bp or more of the geminivirus target site.

After a DSB is introduced, crossing over occur between the geminivirus double stranded genome and the sequences on both the left homology arm and the right homology arm so as to incorporate the first, second, and third nucleic acids into the geminivirus genome by homology-directed repair (HDR).

In various embodiments, the total length of the left homology arm nucleic acid sequence is from 30 bp to 800 bp or more, and may be any integer between 30 and 800 bp. The total length of the right homology arm nucleic acid sequence may be from 30 bp to 800 bp, and may be any integer between 30 and 800 bp. The length of the left homology arm can be from 30 bp to 400 bp, 40 bp to 300 bp, 50 bp to 250 bp, 60 bp to 200 bp, 70 bp to 170 bp, 80 bp to 150 bp, 90 bp to 140 bp, 100 bp to 120 bp. The length of the right homology arm can be from 30 bp to 400 bp, 40 bp to 300 bp, 50 bp to 250 bp, 60 bp to 200 bp, 70 bp to 170 bp, 80 bp to 150 bp, 90 bp to 140 bp, 100 bp to 120 bp. If the left homology arm and the right homology arm flank the first, second, third and optional fourth nucleic acid sequences, the nucleic acid comprising the first, second and third nucleic acid sequences can be introduced into a geminivirus genome at or near the geminivirus target site by homologous recombination.

In some embodiments, the first, second, third and fourth nucleic acid sequences are operably linked to a constitutive promoter, an inducible promoter, or a plant tissue specific promoter. Exemplary plant tissue specific promoters include egg apparatus-specific enhancer (EASE), cruciferin, napin, or rubisco small subunit promoter, and promoters that are activated by alternative splicing of a suicide exon. Exemplary constitutive promoters include constitutive RNA pol II promoters such as the 35S, Nos-P, and ubiquitin promoters, and constitutive RNA pol III promoters such as the U6 promoter. Examples of inducible promoters include the virion-sense promoter from geminivirus, and the XVE promoter. In some embodiments, for example, a Cas coding sequence can be operably linked to an inducible XVE promoter, which can be activated by estradiol. Expression of the first, second, third and fourth nucleic acids in a plant can be activated by treating the plant with estradiol, and the expressed protein then can cleave geminivirus DNA at the target sequence.

In some embodiments, each of the first, second, third and fourth nucleic acid sequences can have its own promoter. In other embodiments, a polycistronic approach can be used to express multiple nucleic acid sequences from one promoter. For example, multiple crRNAs can be expressed from a single promoter, along the lines of the bacterial pre-crRNA molecule, while the tracrRNA can be expressed from a separate promoter. In some cases, a polycistronic message can include one or more crRNA sequences and one or more tracrRNA sequences, or two or gRNA sequences. If the SDN protein is Cpf1, a polycistronic message containing two or more crRNA sequences can be expressed that will be processed by Cpf1 into mature crRNA molecules that function as guides for the Cpf1 protein (see Zetsche et al., 2016).

In some embodiments, portions of the geminivirus genome that include symptom-modulating DNA satellites associated with geminiviruses are targeted for DSB. An example of a DNA beta molecule is the cotton leaf curl virus beta (ACCGTGGGCGAGCGGTGTCTTTGGCGTTCCATGTGGGTCCCACAATATCCA AAAGAAGAATAATGGACTGGGTCAATGCAATTGGGCCTTAAATGAAATGGG CTTGGACCAGTAGATTCGAGACTGGGCCAATAGAATAAAACAACAAATGG ACTCATAATCAAAACAAAGTGTTTATTCATGTCAAATACATTACACACTCAC ACACACACAGTCGTACACACATCATATTCATCCCCTATACGTATATCAACTAA TGGGGCCTCATGCATCATCATTATATCAATAGCCTCTACCATGTCCTCCTGGC GAAAGTCCCGAACATGACAATCCCTATACATGATCTTTAATATATTATGTATC CCTTCCTCCAAATTGTTGAAGTCGAAAGGCGGTATGATCCCATCATGGCCGT ATGGGATCATGAAGGTCTTCTTTGCCAGGGCAGGTGATCTTGTTGAGCACA ATTCAATCCGCACAAGGATTGAATTGTCCTCGTGGATCTTCACGTCGACGGT AAATACCATCCCCTTCTCGTTAGTATACTTGATTGTCATTTGCATTTAATTATG AACAACACATGAGATGAATCTTCTTAAATAGCGTCCATATATTTGGATATATG GACATAATGCATATACGTGGTGCAATAATTATCATATGAATATGAGTGGAGA CATATATGATTGATATCTACAGGACTATCAATCATCGTCAAGAAAAAAGGGA T AAAAGAAACCTTATTCATGAAAGGATTAGGTAGGGAAAAATAAAAGAAGG AAATTAAGGAAAAGAAAACCCACAATGAAATAATTAAGAAAAAAAAAAGA AAAAAAGAACACAAGAAAGGAAAACAGAAACATGAGCACAAACAGAAA CCTCTTGAGAAAATATTGGAGCGCAGCGGGAAAACCAAGAAAAACAAACC AAGGAAGACACTTGAGCAAAAAGGGAAAACACAAACTAAACTAGGAGGG TCCAACATGAAATTATGAAAAGTCCGTACACAGTAATTAATCATTAATTACT GCGCAGTAAATGTGAATAAAATTAACCCGAAGGGTTAATTTTCGAGACCCC GATAGGTAATTGAGTCCCCAATATATCGGGGACTCAATCGGGGTCATGAGA GAGAAATAAATCCCGGATACCGAAACTACCCTCAAAGCTGTGTCTGGAAGG CGCGTGGGAGTGCGCTGAAAAAGGTGACCTTCTCTCTCCAAAAACTCACC GGAACGGCCAAACTGGCTGATTCCGGCATCTAATCACGACACGCGCGGCG GTGTGTACCCCTGGGAGGGTAGACCACTACGCTACGCAGCAGCCTTAGCTA CGCCGGAGCTTAGCTCGCCCACGTTCTAATATT) (SEQ ID NO: 30). Also, the CRISPR/SDN technology can be multiplexed, enabling the targeting of multiple different regions on the same geminivirus, or on multiple geminivirus species or strains. Targeting multiple regions of the same virus has the benefit of potentially increasing the durability of resistance. Targeting regions on multiple virus species has the benefit of increasing the broadness of resistance.

In various embodiments, the geminivirus target site is within a critical region of the genome required for virus biology. The critical region may be the Rep protein open reading frame (ORF), the movement protein ORF, the coat protein ORF, or the hairpin region that mediates the origin of replication. Any portion of these critical regions can serve as the target site.

In various embodiments, the one or more crRNA sequences and the tracrRNA sequence are stably integrated into the genome of the plant cell. Integration can occur by using Agrobacterium-mediated transfer of nucleic acids sequences into the plant. In various embodiments, the Agrobacterium is transformed with the first, second, third and fourth nucleic acids. The transformed Agrobacterium can effectively integrate the first, second, third and fourth nucleic acids into the plant genome by treating plants with the Agrobacterium.

After a plant or plant cell is infected or transfected with nucleic acids encoding the DNA protein and the crRNA and tracrRNA sequences, any suitable method can be used to determine whether the first, second, third and optionally fourth nucleic acid sequences have integrated into the genome of the plant or plant cell. For example, thermal asymmetric interlaced polymerase chain reaction (PCR) or Southern blotting of genomic DNA from a potentially transgenic plant, plant part, or plant cell, or from progeny thereof, can be used to assess whether integration has occurred. In some embodiments, for example, Western blotting of cellular extracts can be used to determine whether the SDN protein is present, and Northern blotting of cellular RNA can be used to determine whether the crRNA and tracrRNA are expressed.

After it has been determined that a transgenic plant, plant part, or plant cell contain the first, second, third and optionally fourth nucleic acid sequences, and the SDN and crRNA and tracrRNA sequences are expressed, any suitable method(s) can be used to propagate the plant, plant part, or plant cell to generate a population of transgenic plants that express the these nucleic acids and thus have increased geminivirus resistance.

In addition to the methods described herein, also disclosed are plants, plant parts, and plant cells prepared by any of the methods and embodiments thereof described herein. Such plants, plant parts, and plant cells have increased geminivirus resistance when the CRISPR/Cas components are expressed.

### Additional Embodiments

The following additional embodiments are described herein.

Embodiment 1 is a method for generating a plant cell having the potential for increased resistance to geminivirus infection. The method comprises transforming the plant cell with a nucleic acid comprising:

(i) a nucleic acid sequence that encodes a Clustered Regularly Interspaced Short Palindromic Repeats-associated (system) Cas protein capable of modifying DNA by introducing a double stranded break,

(ii) one or more Clustered Regularly Interspaced Short Palindromic Repeats (CRISPR) RNA (crRNA) nucleic acid sequences comprising sequence complementary to a sequence from a geminivirus target site, where the geminivirus target site is within one or more geminivirus sequences required for replication or packaging,

(iii) a trans-activating crRNA (tracrRNA) nucleic acid sequence, and

(iv) a left homology arm nucleic acid sequence and a right homology arm nucleic acid sequence, each comprising a nucleotide sequence from the geminivirus genome that is adjacent to the geminivirus target site, and where the left homology arm nucleic acid sequence and the right homology arm nucleic acid sequence are effective to introduce nucleic acid sequences (i), (ii), and/or (iii) into a geminivirus genome at or near the geminivirus target site by homologous recombination.

The nucleic acid is stably integrated into the genome of the plant cell.

Optionally in embodiment 1, the (i) nucleic acid sequence that encodes the Cas protein, the (ii) one or more crRNA nucleic acid sequences, and the (iii) tracrRNA nucleic acid sequence are flanked by the left homology arm nucleic acid sequence and the right homology arm nucleic acid sequence.

Optionally in embodiment 1, upon exposure of the plant cell to a geminivirus, the Cas protein mediates a double stranded break in the geminivirus genome at or near the geminivirus target site, and the nucleic acid sequences (i), (ii), and (iii) are inserted into the geminivirus genome at the double stranded break by homologous recombination between the nucleic acid and the geminivirus genome to form a modified geminivirus.

Optionally in embodiment 1, the modified geminivirus is unable to undergo one or more of replication, packaging, transport from the plant cell, or infection of a plant cell.

Optionally in embodiment 1, the left homology arm is at least 30 bp, optionally at least 400 bp, and the right homology arm is at least 30 bp, optionally at least 400 bp.

Optionally in embodiment 1, the sequence contained within the geminivirus genome required for geminivirus replication or packaging is a Rep protein ORF, a movement protein ORF, a coat protein protein ORF, a hairpin region that mediates replication origin, or a satellite DNA.

Optionally in embodiment 1, each of the (ii) one or more crRNA sequences is fused to the (iii) tracrRNA sequence.

Optionally in embodiment 1, the nucleic acid is operably linked to a constitutive promoter, an inducible promoter or a plant tissue specific promoter.

Optionally in embodiment 1, the plant cell is in a plant.

Optionally in embodiment 1, the transforming comprises Agrobacterium-mediated transformation, electroporation transformation, polyethylene glycol (PEG) transformation, or biolistic transformation.

Embodiment 2 is a method for generating a plant cell having the potential for increased resistance to geminivirus infection. The method comprises transforming the plant cell with a nucleic acid comprising:

(i) a nucleic acid sequence that encodes a Clustered Regularly Interspaced Short Palindromic Repeats-associated (system) Cas protein capable of modifying DNA by introducing a double stranded break,

(ii) one or more Clustered Regularly Interspaced Short Palindromic Repeats (CRISPR) RNA (crRNA) nucleic acid sequences comprising sequence complementary to a sequence from a geminivirus target site, where the geminivirus target site is within one or more geminivirus sequences required for replication or packaging,

(iii) a trans-activating crRNA (tracrRNA) nucleic acid sequence, and

(iv) a left homology arm nucleic acid sequence and a right homology arm nucleic acid sequence, each comprising a nucleotide sequence from the geminivirus genome that is adjacent to the geminivirus target site, and where the left homology arm nucleic acid sequence and the right homology arm nucleic acid sequence are effective to introduce nucleic acid sequence (i), (ii), or (iii) into a geminivirus genome at or near the geminivirus target site by homologous recombination.

The nucleic acid is stably integrated into the genome of the plant cell.

Optionally in embodiment 2, the (i) nucleic acid sequence that encodes the Cas protein is flanked by the left homology arm nucleic acid sequence and the right homology arm nucleic acid sequence.

Optionally in embodiment 2, the (ii) one or more crRNA nucleic acid sequences is flanked by the left homology arm nucleic acid sequence and the right homology arm nucleic acid sequence.

Optionally in embodiment 2, the (iii) tracrRNA nucleic acid sequence is flanked by the left homology arm nucleic acid sequence and the right homology arm nucleic acid sequence.

Optionally in embodiment 2, upon exposure of the plant cell to a geminivirus, the Cas protein mediates a double stranded break in the geminivirus genome at or near the geminivirus target site, and at least one of the nucleic acid sequences (i), (ii), and (iii) are inserted into the geminivirus genome at the double stranded break by homologous recombination between the nucleic acid and the geminivirus genome to form a modified geminivirus. The modified geminivirus is unable to undergo one or more of replication, packaging transport from the plant cell, or infection of new plant cells.

Optionally in embodiment 2, the left homology arm is at least 30 bp, optionally at least 400 bp, and the right homology arm is at least 30 bp, optionally at least 400 bp.

Optionally in embodiment 2, the sequence contained within the geminivirus genome required for geminivirus replication or packaging is a Rep protein ORF, a movement protein ORF, a coat protein protein ORF, a hairpin region that mediates replication origin, or a satellite DNA.

Optionally in embodiment 2, the insert sequence comprises one or more of a sequence that encodes a Clustered Regularly Interspaced Short Palindromic Repeats-associated Cas protein, a crRNA sequence, and a tracrRNA sequence targeted to one or more geminivirus sequences.

Optionally in embodiment 2, each of the (ii) one or more crRNA sequences is fused to the (iii) tracrRNA sequence.

Optionally in embodiment 2, the nucleic acid is operably linked to a constitutive promoter, an inducible promoter or a plant tissue specific promoter.

Optionally in embodiment 2, the plant cell is in a plant, and where the transforming comprises Agrobacterium-mediated transformation, electroporation transformation, polyethylene glycol (PEG) transformation, or biolistic transformation.

Embodiment 3 is a method for generating a plant cell having the potential for increased resistance to geminivirus infection. The method comprises transforming the plant cell with a nucleic acid comprising:

(i) a nucleic acid sequence that encodes a Clustered Regularly Interspaced Short Palindromic Repeats-associated Cas protein capable of modifying DNA by introducing a double stranded break,

(ii) one or more Clustered Regularly Interspaced Short Palindromic Repeats (CRISPR) RNA (crRNA) nucleic acid sequences comprising sequence complementary to a sequence from a geminivirus target site, where the geminivirus target site is within one or more geminivirus sequences required for replication or packaging,

(iii) a trans-activating crRNA (tracrRNA) nucleic acid sequence,

(iv) an insert nucleic acid sequence, and

(v) a left homology arm nucleic acid sequence and a right homology arm nucleic acid sequence, each comprising a nucleotide sequence from the geminivirus genome that is adjacent to the geminivirus target site, and where the left homology arm nucleic acid sequence and the right homology arm nucleic acid sequence flank the (iv) insert nucleic acid sequence and are effective to insert the (iv) insert nucleic acid sequence into a geminivirus genome at or near the geminivirus target site by homologous recombination.

The nucleic acid is stably integrated into the genome of the plant cell.

Optionally in embodiment 3, the (iv) insert nucleic acid sequence comprises one or more of the (i) nucleic acid sequence that encodes the Cas protein, the (ii) one or more crRNA nucleic acid sequences, and the (iii) tracrRNA nucleic acid sequence.

Optionally in embodiment 3, the (iv) insert nucleic acid sequence comprises the (i) nucleic acid sequence that encodes the Cas protein, the (ii) one or more crRNA nucleic acid sequences, and the (iii) tracrRNA nucleic acid sequence.

Optionally in embodiment 3, the insert sequence is at least 200 bp, optionally from 200 bp to 9 kb.

Optionally in embodiment 3, upon exposure of the plant cell to a geminivirus, the Cas protein mediates a double stranded break in the geminivirus genome at or near the geminivirus target site, and the (iv) insert nucleic acid sequence is inserted into the geminivirus genome at the double stranded break by homologous recombination. The modified geminivirus is unable to undergo one or more of replication, packaging or transport from the plant cell.

Optionally in embodiment 3, the left homology arm is at least 30 bp, optionally at least 400 bp, and the right homology arm is at least 30 bp, optionally at least 400 bp. The sequence contained within the geminivirus genome required for geminivirus replication or packaging may be a Rep protein ORF, a movement protein ORF, a coat protein ORF, a hairpin region that mediates replication origin, or a satellite DNA. The insert sequence may comprise one or more of a sequence that encodes a Clustered Regularly Interspaced Short Palindromic Repeats-associated Cas protein, a crRNA sequence, and a tracrRNA sequence targeted to one or more geminivirus sequences.

Optionally in embodiment 3, each of the (ii) one or more crRNA sequences is fused to the (iii) tracrRNA sequence.

Optionally in embodiment 3, the nucleic acid is operably linked to a constitutive promoter, an inducible promoter or a plant tissue specific promoter.

Optionally in embodiment 3, the plant cell is in a plant. Transforming can occur by any of Agrobacterium-mediated transformation, electroporation transformation, polyethylene glycol (PEG) transformation, or biolistic transformation.

Embodiment 4 is a plant, plant part, or plant cell that has increased resistance to geminivirus infection, where the genome of the plant, plant part, or plant cell comprises a nucleic acid comprising:

(i) a nucleic acid sequence that encodes a Clustered Regularly Interspaced Short Palindromic Repeats-associated (system) Cas protein capable of modifying DNA by introducing a double stranded break,

(ii) one or more Clustered Regularly Interspaced Short Palindromic Repeats (CRISPR) RNA (crRNA) nucleic acid sequences comprising sequence complementary to a sequence from a geminivirus target site, where the geminivirus target site is within one or more geminivirus sequences required for replication or packaging,

(iii) a trans-activating crRNA (tracrRNA) nucleic acid sequence, and

(iv) a left homology arm nucleic acid sequence and a right homology arm nucleic acid sequence, each comprising a nucleotide sequence from the geminivirus genome that is adjacent to the geminivirus target site, and where the left homology arm nucleic acid sequence and the right homology arm nucleic acid sequence are effective to introduce nucleic acid sequences (i), (ii), and/or (iii) into a geminivirus genome at or near the geminivirus target site by homologous recombination.

The nucleic acid is stably integrated into the genome of the plant cell.

Optionally in embodiment 4, the (i) nucleic acid sequence that encodes the Cas protein, the (ii) one or more crRNA nucleic acid sequences, and the (iii) tracrRNA nucleic acid sequence are flanked by the left homology arm nucleic acid sequence and the right homology arm nucleic acid sequence.

Optionally in embodiment 4, upon exposure of the plant cell to a geminivirus,

(a) the Cas protein mediates a double stranded break in the geminivirus genome at or near the geminivirus target site, and

(b) the nucleic acid sequences (i), (ii), and/or (iii) are inserted into the geminivirus genome at the double stranded break by homologous recombination between the nucleic acid and the geminivirus genome to form a modified geminivirus.

The modified geminivirus is unable to undergo one or more of replication, packaging or transport from the plant cell.

Optionally in embodiment 4, the plant, plant part, or plant cell of claim 31, where the left homology arm is at least 30 bp, optionally at least 400 bp, and the right homology arm is at least 30 bp, optionally at least 400 bp.

Optionally in embodiment 4, the sequence contained within the geminivirus genome required for geminivirus replication or packaging is a Rep protein ORF, a movement protein ORF, a coat protein protein ORF, a hairpin region that mediates replication origin, or a satellite DNA.

Optionally in embodiment 4, each of the (ii) one or more crRNA sequences is fused to the (iii) tracrRNA sequence.

Optionally in embodiment 4, the nucleic acid is operably linked to a constitutive promoter, an inducible promoter or a plant tissue specific promoter.

Optionally in embodiment 4, the plant cell is in a plant. Transforming occurs by any of Agrobacterium-mediated transformation, electroporation transformation, polyethylene glycol (PEG) transformation, and biolistic transformation.

Embodiment 5 is a plant, plant part, or plant cell that has increased resistance to geminivirus infection. The genome of the plant, plant part, or plant cell comprises a nucleic acid comprising:

(i) a nucleic acid sequence that encodes a Clustered Regularly Interspaced Short Palindromic Repeats-associated (system) Cas protein capable of modifying DNA by introducing a double stranded break,

(ii) one or more Clustered Regularly Interspaced Short Palindromic Repeats (CRISPR) RNA (crRNA) nucleic acid sequences comprising sequence complementary to a sequence from a geminivirus target site, where the geminivirus target site is within one or more geminivirus sequences required for replication or packaging,

(iii) a trans-activating crRNA (tracrRNA) nucleic acid sequence, and

(iv) a left homology arm nucleic acid sequence and a right homology arm nucleic acid sequence, each comprising a nucleotide sequence from the geminivirus genome that is adjacent to the geminivirus target site, and where the left homology arm nucleic acid sequence and the right homology arm nucleic acid sequence are effective to introduce nucleic acid sequence (i), (ii), or (iii) into a geminivirus genome at or near the geminivirus target site by homologous recombination.

The nucleic acid is stably integrated into the genome of the plant cell.

Optionally in embodiment 5, the (i) nucleic acid sequence that encodes the Cas protein is flanked by the left homology arm nucleic acid sequence and the right homology arm nucleic acid sequence.

Optionally in embodiment 5, the (ii) one or more crRNA nucleic acid sequences is flanked by the left homology arm nucleic acid sequence and the right homology arm nucleic acid sequence.

Optionally in embodiment 5, the (iii) tracrRNA nucleic acid sequence is flanked by the left homology arm nucleic acid sequence and the right homology arm nucleic acid sequence.

Optionally in embodiment 5, upon exposure of the plant cell to a geminivirus, the Cas protein mediates a double stranded break in the geminivirus genome at or near the geminivirus target site, and at least one of the nucleic acid sequences (i), (ii), and (iii) are inserted into the geminivirus genome at the double stranded break by homologous recombination between the nucleic acid and the geminivirus genome to form a modified geminivirus. The modified geminivirus is unable to undergo one or more of replication, packaging or transport from the plant cell.

Optionally in embodiment 5, the left homology arm is at least 30 bp, optionally at least 400 bp, and the right homology arm is at least 30 bp, optionally at least 400 bp.

Optionally in embodiment 5, the sequence contained within the geminivirus genome required for geminivirus replication or packaging is a Rep protein ORF, a movement protein ORF, a coat protein protein ORF, a hairpin region that mediates replication origin, or a satellite DNA.

Optionally in embodiment 5, the insert sequence comprises one or more of a sequence that encodes a Clustered Regularly Interspaced Short Palindromic Repeats-associated Cas protein, a crRNA sequence, and a tracrRNA sequence targeted to one or more geminivirus sequences.

Optionally in embodiment 5, each of the (ii) one or more crRNA sequences is fused to the (iii) tracrRNA sequence.

Optionally in embodiment 5, the nucleic acid is operably linked to a constitutive promoter, an inducible promoter or a plant tissue specific promoter.

Optionally in embodiment 5, the plant cell is in a plant, and where the transforming comprises Agrobacterium-mediated transformation, electroporation transformation, polyethylene glycol (PEG) transformation, or biolistic transformation.

Embodiment 6 is a plant, plant part, or plant cell that has increased resistance to geminivirus infection. The genome of the plant, plant part, or plant cell comprises a nucleic acid comprising:

(i) a nucleic acid sequence that encodes a Clustered Regularly Interspaced Short Palindromic Repeats-associated (system) Cas protein capable of modifying DNA by introducing a double stranded break,

(ii) one or more Clustered Regularly Interspaced Short Palindromic Repeats (CRISPR) RNA (crRNA) nucleic acid sequences comprising sequence complementary to a sequence from a geminivirus target site, where the geminivirus target site is within one or more geminivirus sequences required for replication or packaging,

(iii) a trans-activating crRNA (tracrRNA) nucleic acid sequence,

(iv) an insert nucleic acid sequence, and

(v) a left homology arm nucleic acid sequence and a right homology arm nucleic acid sequence, each comprising a nucleotide sequence from the geminivirus genome that is adjacent to the geminivirus target site, and where the left homology arm nucleic acid sequence and the right homology arm nucleic acid sequence flank the (iv) insert nucleic acid sequence and are effective to insert the (iv) insert nucleic acid sequence into a geminivirus genome at or near the geminivirus target site by homologous recombination.

The nucleic acid is stably integrated into the genome of the plant cell.

Optionally in embodiment 6, the (iv) insert nucleic acid sequence comprises one or more of the (i) nucleic acid sequence that encodes the Cas protein, the (ii) one or more crRNA nucleic acid sequences, and the (iii) tracrRNA nucleic acid sequence.

Optionally in embodiment 6, the (iv) insert nucleic acid sequence comprises the (i) nucleic acid sequence that encodes the Cas protein, the (ii) one or more crRNA nucleic acid sequences, and the (iii) tracrRNA nucleic acid sequence.

Optionally in embodiment 6, the (iv) insert sequence is at least 200 bp, optionally from 200 bp to 9 kb.

Optionally in embodiment 6, upon exposure of the plant cell to a geminivirus, the Cas protein mediates a double stranded break in the geminivirus genome at or near the geminivirus target site, and the (iv) insert nucleic acid sequence is inserted into the geminivirus genome at the double stranded break by homologous recombination. The modified geminivirus is unable to undergo one or more of replication, packaging or transport from the plant cell.

Optionally in embodiment 6, the left homology arm is at least 30 bp, optionally at least 400 bp, and the right homology arm is at least 30 bp, optionally at least 400 bp.

Optionally in embodiment 6, the sequence contained within the geminivirus genome required for geminivirus replication or packaging is a Rep protein ORF, a movement protein ORF, a coat protein protein ORF, a hairpin region that mediates replication origin, or a satellite DNA.

Optionally in embodiment 6, the insert sequence comprises one or more of a sequence that encodes a Clustered Regularly Interspaced Short Palindromic Repeats-associated Cas protein, a crRNA sequence, and a tracrRNA sequence targeted to one or more geminivirus sequences.

Optionally in embodiment 6, each of the (ii) one or more crRNA sequences is fused to the (iii) tracrRNA sequence.

Optionally in embodiment 6, the nucleic acid is operably linked to a constitutive promoter, an inducible promoter or a plant tissue specific promoter.

Optionally in embodiment 6, the plant cell is in a plant, and the transforming comprises Agrobacterium-mediated transformation, electroporation transformation, polyethylene glycol (PEG) transformation, or biolistic transformation.

Embodiment 7 is a method for generating a plant cell having the potential for increased resistance to geminivirus infection. The method comprises transforming the plant cell with a nucleic acid comprising:

(i) a nucleic acid sequence that encodes a Cpf1 protein capable of modifying DNA by introducing a double stranded break,

(ii) one or more guide RNA (gRNA) nucleic acid sequences, where the gRNA nucleic acid sequence further comprises nucleic acid sequence complementary to a sequence from a geminivirus target site, where the geminivirus target site is within one or more geminivirus sequences required for replication or packaging, and

(iii) a left homology arm nucleic acid sequence and a right homology arm nucleic acid sequence, each comprising a nucleotide sequence from the geminivirus genome that is adjacent to the geminivirus target site, and where the left homology arm nucleic acid sequence and the right homology arm nucleic acid sequence are effective to introduce one or more of nucleic acid sequences (i) and (ii) into a geminivirus genome at or near the geminivirus target site by homologous recombination.

The nucleic acid is stably integrated into the genome of the plant cell.

The invention will be further described in the following examples, which do not limit the scope of the invention described in the claims.

### EXAMPLES

The following examples describe the various aspects and embodiments described above. However, the use of these and other examples anywhere in the specification is illustrative only and in no way limits the scope and meaning of any of the disclosure or of any exemplified term. Likewise, any claimed subject matter is not limited to any particular preferred embodiments described here. Indeed, many modifications and variations may be apparent to those skilled in the art upon reading this specification, and such variations can be made without departing in spirit or in scope from the aspects and embodiments disclosed herein. Any claimed subject matter is therefore to be limited only by the terms of the appended claims along with the full scope of equivalents to which those claims are entitled.

### Example 1

### Plasmid Encoding Cas9 for Transfection into Plants

Plasmid DNA is modified to encode a nucleic acid comprising a Cas9 protein. The plasmid DNA contains a nuclease-active, plant codon-optimized Cas9 sequence that includes at least one translationally fused nuclear-localization signal peptide , downstream of and expressed by a constitutive RNA pol II promoter. The plasmid also contains selectable markers effective to provide Agrobacterum tumefaciens resistance to both kanamycin and gentamicin, and T-DNA borders suitable for causing transfer of the included sequence between the borders into the plant cell.

The following Streptococcus pyogenes Cas9-encoding nucleotide sequence is amplified by PCR and subcloned into the MCS:

(SEQ ID NO: 31)
ATGGATAAGAAATACTCAATAGGCTTAGATATCGGCACAAATAGCGTCGG
ATGGGCGGTGATCACTGATGAATATAAGGTTCCGTCTAAAAAGTTCAAGG
TTCTGGGAAATACAGACCGCCACAGTATCAAAAAAAATCTTATAGGGGCT
CTTTTATTTGACAGTGGAGAGACAGCGGAAGCGACTCGTCTCAAACGGAC
AGCTCGTAGAAGGTATACACGTCGGAAGAATCGTATTTGTTATCTACAGG
AGATTTTTTCAAATGAGATGGCGAAAGTAGATGATAGTTTCTTTCATCGA
CTTGAAGAGTCTTTTTTGGTGGAAGAAGACAAGAAGCATGAACGTCATCC
TATTTTTGGAAATATAGTAGATGAAGTTGCTTATCATGAGAAATATCCAA
CTATCTATCATCTGCGAAAAAAATTGGTAGATTCTACTGATAAAGCGGAT
TTGCGCTTAATCTATTTGGCCTTAGCGCATATGATTAAGTTTCGTGGTCA
TTTTTTGATTGAGGGAGATTTAAATCCTGATAATAGTGATGTGGACAAAC
TATTTATCCAGTTGGTACAAACCTACAATCAATTATTTGAAGAAAACCCT
ATTAACGCAAGTGGAGTAGATGCTAAAGCGATTCTTTCTGCACGATTGAG
TAAATCAAGACGATTAGAAAATCTCATTGCTCAGCTCCCCGGTGAGAAGA
AAAATGGCTTATTTGGGAATCTCATTGCTTTGTCATTGGGTTTGACCCCT
AATTTTAAATCAAATTTTGATTTGGCAGAAGATGCTAAATTACAGCTTTC
AAAAGATACTTACGATGATGATTTAGATAATTTATTGGCGCAAATTGGAG
ATCAATATGCTGATTTGTTTTTGGCAGCTAAGAATTTATCAGATGCTATT
TTACTTTCAGATATCCTAAGAGTAAATACTGAAATAACTAAGGCTCCCCT
ATCAGCTTCAATGATTAAACGCTACGATGAACATCATCAAGACTTGACTC
TTTTAAAAGCTTTAGTTCGACAACAACTTCCAGAAAAGTATAAAGAAATC
TTTTTTGATCAATCAAAAAACGGATATGCAGGTTATATTGATGGGGGAGC
TAGCCAAGAAGAATTTTATAAATTTATCAAACCAATTTTAGAAAAAATGG
ATGGTACTGAGGAATTATTGGTGAAACTAAATCGTGAAGATTTGCTGCGC
AAGCAACGGACCTTTGACAACGGCTCTATTCCCCATCAAATTCACTTGGG
TGAGCTGCATGCTATTTTGAGAAGACAAGAAGACTTTTATCCATTTTTAA
AAGACAATCGTGAGAAGATTGAAAAAATCTTGACTTTTCGAATTCCTTAT
TATGTTGGTCCATTGGCGCGTGGCAATAGTCGTTTTGCATGGATGACTCG
GAAGTCTGAAGAAACAATTACCCCATGGAATTTTGAAGAAGTTGTCGATA
AAGGTGCTTCAGCTCAATCATTTATTGAACGCATGACAAACTTTGATAAA
AATCTTCCAAATGAAAAAGTACTACCAAAACATAGTTTGCTTTATGAGTA
TTTTACGGTTTATAACGAATTGACAAAGGTCAAATATGTTACTGAAGGAA
TGCGAAAACCAGCATTTCTTTCAGGTGAACAGAAGAAAGCCATTGTTGAT
TTACTCTTCAAAACAAATCGAAAAGTAACCGTTAAGCAATTAAAAGAAGA
TTATTTCAAAAAAATAGAATGTTTTGATAGTGTTGAAATTTCAGGAGTTG
AAGATAGATTTAATGCTTCATTAGGTACCTACCATGATTTGCTAAAAATT
ATTAAAGATAAAGATTTTTTGGATAATGAAGAAAATGAAGATATCTTAGA
GGATATTGTTTTAACATTGACCTTATTTGAAGATAGGGAGATGATTGAGG
AAAGACTTAAAACATATGCTCACCTCTTTGATGATAAGGTGATGAAACAG
CTTAAACGTCGCCGTTATACTGGTTGGGGACGTTTGTCTCGAAAATTGAT
TAATGGTATTAGGGATAAGCAATCTGGCAAAACAATATTAGATTTTTTGA
AATCAGATGGTTTTGCCAATCGCAATTTTATGCAGCTGATCCATGATGAT
AGTTTGACATTTAAAGAAGACATTCAAAAAGCACAAGTGTCTGGACAAGG
CGATAGTTTACATGAACATATTGCAAATTTAGCTGGTAGCCCTGCTATTA
AAAAAGGTATTTTACAGACTGTAAAAGTTGTTGATGAATTGGTCAAAGTA
ATGGGGCGGCATAAGCCAGAAAATATCGTTATTGAAATGGCACGTGAAAA
TCAGACAACTCAAAAGGGCCAGAAAAATTCGCGAGAGCGTATGAAACGAA
TCGAAGAAGGTATCAAAGAATTAGGAAGTCAGATTCTTAAAGAGCATCCT
GTTGAAAATACTCAATTGCAAAATGAAAAGCTCTATCTCTATTATCTCCA
AAATGGAAGAGACATGTATGTGGACCAAGAATTAGATATTAATCGTTTAA
GTGATTATGATGTCGATCACATTGTTCCACAAAGTTTCCTTAAAGACGAT
TCAATAGACAATAAGGTCTTAACGCGTTCTGATAAAAATCGTGGTAAATC
GGATAACGTTCCAAGTGAAGAAGTAGTCAAAAAGATGAAAAACTATTGGA
GACAACTTCTAAACGCCAAGTTAATCACTCAACGTAAGTTTGATAATTTA
ACGAAAGCTGAACGTGGAGGTTTGAGTGAACTTGATAAAGCTGGTTTTAT
CAAACGCCAATTGGTTGAAACTCGCCAAATCACTAAGCATGTGGCACAAA
TTTTGGATAGTCGCATGAATACTAAATACGATGAAAATGATAAACTTATT
CGAGAGGTTAAAGTGATTACCTTAAAATCTAAATTAGTTTCTGACTTCCG
AAAAGATTTCCAATTCTATAAAGTACGTGAGATTAACAATTACCATCATG
CCCATGATGCGTATCTAAATGCCGTCGTTGGAACTGCTTTGATTAAGAAA
TATCCAAAACTTGAATCGGAGTTTGTCTATGGTGATTATAAAGTTTATGA
TGTTCGTAAAATGATTGCTAAGTCTGAGCAAGAAATAGGCAAAGCAACCG
CAAAATATTTCTTTTACTCTAATATCATGAACTTCTTCAAAACAGAAATT
ACACTTGCAAATGGAGAGATTCGCAAACGCCCTCTAATCGAAACTAATGG
GGAAACTGGAGAAATTGTCTGGGATAAAGGGCGAGATTTTGCCACAGTGC
GCAAAGTATTGTCCATGCCCCAAGTCAATATTGTCAAGAAAACAGAAGTA
CAGACAGGCGGATTCTCCAAGGAGTCAATTTTACCAAAAAGAAATTCGGA
CAAGCTTATTGCTCGTAAAAAAGACTGGGATCCAAAAAAATATGGTGGTT
TTGATAGTCCAACGGTAGCTTATTCAGTCCTAGTGGTTGCTAAGGTGGAA
AAAGGGAAATCGAAGAAGTTAAAATCCGTTAAAGAGTTACTAGGGATCAC
AATTATGGAAAGAAGTTCCTTTGAAAAAAATCCGATTGACTTTTTAGAAG
CTAAAGGATATAAGGAAGTTAAAAAAGACTTAATCATTAAACTACCTAAA
TATAGTCTTTTTGAGTTAGAAAACGGTCGTAAACGGATGCTGGCTAGTGC
CGGAGAATTACAAAAAGGAAATGAGCTGGCTCTGCCAAGCAAATATGTGA
ATTTTTTATATTTAGCTAGTCATTATGAAAAGTTGAAGGGTAGTCCAGAA
GATAACGAACAAAAACAATTGTTTGTGGAGCAGCATAAGCATTATTTAGA
TGAGATTATTGAGCAAATCAGTGAATTTTCTAAGCGTGTTATTTTAGCAG
ATGCCAATTTAGATAAAGTTCTTAGTGCATATAACAAACATAGAGACAAA
CCAATACGTGAACAAGCAGAAAATATTATTCATTTATTTACGTTGACGAA
TCTTGGAGCTCCCGCTGCTTTTAAATATTTTGATACAACAATTGATCGTA
AACGATATACGTCTACAAAAGAAGTTTTAGATGCCACTCTTATCCATCAA
TCCATCACTGGTCTTTATGAAACACGCATTGATTTGAGTCAGCTAGGAGG
TGACGGTTCTCCCAAGAAGAAGAGGAAAGTCTCGAGCGGTGGAGCTGCAG
GATGAG.

The resulting plasmid is called pCas9. An Agrobacterium tumefaciens strain is transformed with pCas9 using a freeze-thaw method and plated onto LB agar containing 50 μg/mL kanamyscin and 50 μg/mL gentamicin. 5 mL starter cultures with LB broth with 50 μg/mL kanamyscin and 50 μg/mL gentamicin are inoculated with colonies that grow on the LB agar plates. After overnight incubation, the starter culture is used to inoculate 500 mL of LB broth with 50 μg/mL kanamyscin and 50 μg/mL gentamicin are inoculated with colonies that grow on the LB agar plates. An infiltration suspension is formed by pelleting the Agrobacterium cells, resuspending them in 10 mM MES, 150 μM acetosyringone and 10 mM MgCl₂, and incubating at room temperature for four hours.

To deliver pCas9 to individual plant cells, leaves from a N. benthamiania plant 4-6 weeks old are treated with the infiltration suspension containing the pCas9-containing Agrobacterum tumefaciens. Cells with pCas9-derived T-DNA inserted into the genome are regenerated into whole plants.

### Example 2

### Selection of a Target Sequence, Left Homology Arm, and Right Homology Arm

A target sequence is selected for such that if a double stranded break in the geminivirus genome occurs within the target sequence, geminivirus replication, packaging and/or transport is disrupted. Thus, the preferred target sequence is within a region of the geminivirus genome that is critical for geminivirus biology. The selected target sequence will then be used to design a crRNA or gRNA that is effective to target Cas9, in the following examples, or even another reagent such as Cpf1.

In this example, CTGGAGAAGAGCATGATAGTG (SEQ ID NO: 15) is selected as a target sequence from Motif II within the Rep coding sequence of bean yellow dwarf virus (BeYDV-m). The left and right homology arms comprise about 100 bases of sequence from BeYDV-m immediately adjacent to CTGGAGAAGAGCATGATAGTG (SEQ ID NO: 15) in the 5′ and 3′ directions, respectively. For example, the left homology arm has the sequence:

(SEQ ID NO: 32)
ATATGTAATCAAGGACTTGTTTAGAGTTTCTAGCTGGCTGGATATTAGGG
TGATTTCCTTCAAAATCGAAAAAAGAAGGATCCCTAATACAAGGTTTTTT
ATCAA.

The right homology arm has the sequence:

(SEQ ID NO: 33)
GGTAGTGCCATCTTGATGAAGCTCAGAAGCAACACCAAGGAAGAAAATAA
GAAAAGGTGTGAGTTTCTCCCAGAGAAACTGAATAAATCATCTCTTTGAG
AT.

### Example 3

### Preparing a Construct with Cas9 Between the Left Homology Arm and the Right Homology Arm

A construct is prepared that includes the Cas9 coding sequence flanked by both the left and right homology arms, operatively linked to the RNA polymerase II promoter. An exemplary construct comprises the following sequence:

(SEQ ID NO: 34)
ATATGTAATCAAGGACTTGTTTAGAGTTTCTAGCTGGCTGGATATTAGGG
TGATTTCCTTCAAAATCGAAAAAAGAAGGATCCCTAATACAAGGTTTTTT
ATCAAATGGATAAGAAATACTCAATAGGCTTAGATATCGGCACAAATAGC
GTCGGATGGGCGGTGATCACTGATGAATATAAGGTTCCGTCTAAAAAGTT
CAAGGTTCTGGGAAATACAGACCGCCACAGTATCAAAAAAAATCTTATAG
GGGCTCTTTTATTTGACAGTGGAGAGACAGCGGAAGCGACTCGTCTCAAA
CGGACAGCTCGTAGAAGGTATACACGTCGGAAGAATCGTATTTGTTATCT
ACAGGAGATTTTTTCAAATGAGATGGCGAAAGTAGATGATAGTTTCTTTC
ATCGACTTGAAGAGTCTTTTTTGGTGGAAGAAGACAAGAAGCATGAACGT
CATCCTATTTTTGGAAATATAGTAGATGAAGTTGCTTATCATGAGAAATA
TCCAACTATCTATCATCTGCGAAAAAAATTGGTAGATTCTACTGATAAAG
CGGATTTGCGCTTAATCTATTTGGCCTTAGCGCATATGATTAAGTTTCGT
GGTCATTTTTTGATTGAGGGAGATTTAAATCCTGATAATAGTGATGTGGA
CAAACTATTTATCCAGTTGGTACAAACCTACAATCAATTATTTGAAGAAA
ACCCTATTAACGCAAGTGGAGTAGATGCTAAAGCGATTCTTTCTGCACGA
TTGAGTAAATCAAGACGATTAGAAAATCTCATTGCTCAGCTCCCCGGTGA
GAAGAAAAATGGCTTATTTGGGAATCTCATTGCTTTGTCATTGGGTTTGA
CCCCTAATTTTAAATCAAATTTTGATTTGGCAGAAGATGCTAAATTACAG
CTTTCAAAAGATACTTACGATGATGATTTAGATAATTTATTGGCGCAAAT
TGGAGATCAATATGCTGATTTGTTTTTGGCAGCTAAGAATTTATCAGATG
CTATTTTACTTTCAGATATCCTAAGAGTAAATACTGAAATAACTAAGGCT
CCCCTATCAGCTTCAATGATTAAACGCTACGATGAACATCATCAAGACTT
GACTCTTTTAAAAGCTTTAGTTCGACAACAACTTCCAGAAAAGTATAAAG
AAATCTTTTTTGATCAATCAAAAAACGGATATGCAGGTTATATTGATGGG
GGAGCTAGCCAAGAAGAATTTTATAAATTTATCAAACCAATTTTAGAAAA
AATGGATGGTACTGAGGAATTATTGGTGAAACTAAATCGTGAAGATTTGC
TGCGCAAGCAACGGACCTTTGACAACGGCTCTATTCCCCATCAAATTCAC
TTGGGTGAGCTGCATGCTATTTTGAGAAGACAAGAAGACTTTTATCCATT
TTTAAAAGACAATCGTGAGAAGATTGAAAAAATCTTGACTTTTCGAATTC
CTTATTATGTTGGTCCATTGGCGCGTGGCAATAGTCGTTTTGCATGGATG
ACTCGGAAGTCTGAAGAAACAATTACCCCATGGAATTTTGAAGAAGTTGT
CGATAAAGGTGCTTCAGCTCAATCATTTATTGAACGCATGACAAACTTTG
ATAAAAATCTTCCAAATGAAAAAGTACTACCAAAACATAGTTTGCTTTAT
GAGTATTTTACGGTTTATAACGAATTGACAAAGGTCAAATATGTTACTGA
AGGAATGCGAAAACCAGCATTTCTTTCAGGTGAACAGAAGAAAGCCATTG
TTGATTTACTCTTCAAAACAAATCGAAAAGTAACCGTTAAGCAATTAAAA
GAAGATTATTTCAAAAAAATAGAATGTTTTGATAGTGTTGAAATTTCAGG
AGTTGAAGATAGATTTAATGCTTCATTAGGTACCTACCATGATTTGCTAA
AAATTATTAAAGATAAAGATTTTTTGGATAATGAAGAAAATGAAGATATC
TTAGAGGATATTGTTTTAACATTGACCTTATTTGAAGATAGGGAGATGAT
TGAGGAAAGACTTAAAACATATGCTCACCTCTTTGATGATAAGGTGATGA
AACAGCTTAAACGTCGCCGTTATACTGGTTGGGGACGTTTGTCTCGAAAA
TTGATTAATGGTATTAGGGATAAGCAATCTGGCAAAACAATATTAGATTT
TTTGAAATCAGATGGTTTTGCCAATCGCAATTTTATGCAGCTGATCCATG
ATGATAGTTTGACATTTAAAGAAGACATTCAAAAAGCACAAGTGTCTGGA
CAAGGCGATAGTTTACATGAACATATTGCAAATTTAGCTGGTAGCCCTGC
TATTAAAAAAGGTATTTTACAGACTGTAAAAGTTGTTGATGAATTGGTCA
AAGTAATGGGGCGGCATAAGCCAGAAAATATCGTTATTGAAATGGCACGT
GAAAATCAGACAACTCAAAAGGGCCAGAAAAATTCGCGAGAGCGTATGAA
ACGAATCGAAGAAGGTATCAAAGAATTAGGAAGTCAGATTCTTAAAGAGC
ATCCTGTTGAAAATACTCAATTGCAAAATGAAAAGCTCTATCTCTATTAT
CTCCAAAATGGAAGAGACATGTATGTGGACCAAGAATTAGATATTAATCG
TTTAAGTGATTATGATGTCGATCACATTGTTCCACAAAGTTTCCTTAAAG
ACGATTCAATAGACAATAAGGTCTTAACGCGTTCTGATAAAAATCGTGGT
AAATCGGATAACGTTCCAAGTGAAGAAGTAGTCAAAAAGATGAAAAACTA
TTGGAGACAACTTCTAAACGCCAAGTTAATCACTCAACGTAAGTTTGATA
ATTTAACGAAAGCTGAACGTGGAGGTTTGAGTGAACTTGATAAAGCTGGT
TTTATCAAACGCCAATTGGTTGAAACTCGCCAAATCACTAAGCATGTGGC
ACAAATTTTGGATAGTCGCATGAATACTAAATACGATGAAAATGATAAAC
TTATTCGAGAGGTTAAAGTGATTACCTTAAAATCTAAATTAGTTTCTGAC
TTCCGAAAAGATTTCCAATTCTATAAAGTACGTGAGATTAACAATTACCA
TCATGCCCATGATGCGTATCTAAATGCCGTCGTTGGAACTGCTTTGATTA
AGAAATATCCAAAACTTGAATCGGAGTTTGTCTATGGTGATTATAAAGTT
TATGATGTTCGTAAAATGATTGCTAAGTCTGAGCAAGAAATAGGCAAAGC
AACCGCAAAATATTTCTTTTACTCTAATATCATGAACTTCTTCAAAACAG
AAATTACACTTGCAAATGGAGAGATTCGCAAACGCCCTCTAATCGAAACT
AATGGGGAAACTGGAGAAATTGTCTGGGATAAAGGGCGAGATTTTGCCAC
AGTGCGCAAAGTATTGTCCATGCCCCAAGTCAATATTGTCAAGAAAACAG
AAGTACAGACAGGCGGATTCTCCAAGGAGTCAATTTTACCAAAAAGAAAT
TCGGACAAGCTTATTGCTCGTAAAAAAGACTGGGATCCAAAAAAATATGG
TGGTTTTGATAGTCCAACGGTAGCTTATTCAGTCCTAGTGGTTGCTAAGG
TGGAAAAAGGGAAATCGAAGAAGTTAAAATCCGTTAAAGAGTTACTAGGG
ATCACAATTATGGAAAGAAGTTCCTTTGAAAAAAATCCGATTGACTTTTT
AGAAGCTAAAGGATATAAGGAAGTTAAAAAAGACTTAATCATTAAACTAC
CTAAATATAGTCTTTTTGAGTTAGAAAACGGTCGTAAACGGATGCTGGCT
AGTGCCGGAGAATTACAAAAAGGAAATGAGCTGGCTCTGCCAAGCAAATA
TGTGAATTTTTTATATTTAGCTAGTCATTATGAAAAGTTGAAGGGTAGTC
CAGAAGATAACGAACAAAAACAATTGTTTGTGGAGCAGCATAAGCATTAT
TTAGATGAGATTATTGAGCAAATCAGTGAATTTTCTAAGCGTGTTATTTT
AGCAGATGCCAATTTAGATAAAGTTCTTAGTGCATATAACAAACATAGAG
ACAAACCAATACGTGAACAAGCAGAAAATATTATTCATTTATTTACGTTG
ACGAATCTTGGAGCTCCCGCTGCTTTTAAATATTTTGATACAACAATTGA
TCGTAAACGATATACGTCTACAAAAGAAGTTTTAGATGCCACTCTTATCC
ATCAATCCATCACTGGTCTTTATGAAACACGCATTGATTTGAGTCAGCTA
GGAGGTGACGGTTCTCCCAAGAAGAAGAGGAAAGTCTCGAGCGGTGGAGC
TGCAGGATGAGgGTAGTGCCATCTTGATGAAGCTCAGAAGCAACACCAAG
GAAGAAAATAAGAAAAGGTGTGAGTTTCTCCCAGAGAAACTGAATAAATC
ATCTCTTTGAGAT.

The left and right homology arms from Example 2 are used and are underlined.

### Example 4

### Preparing a Construct Comprising crRNA and tracrRNA

The target sequence of Example 2 is used: CTGGAGAAGAGCATGATAGTG (SEQ ID NO: 15) from Motif II within the Rep coding sequence of bean yellow dwarf virus (BeYDV-m).

With this target sequence, the nucleic acid encoding a crRNA comprises the sequence AATTTCTACTCTTGTAGATCTGGAGAAGAGCATGATAGTG (SEQ ID NO: 35). The nucleic acid encoding a tracrRNA from Streptococcus pyogenes is GTTGGAACCATTCAAAACAGCATAGCAAGTTAAAATAAGGCTAGTCCGTTAT CAACTTGAAAAAGTGGCACCGAGTCGGTGCTTTTT (SEQ ID NO: 36).

A construct is prepared with each of the crRNA and tracrRNA sequences operatively linked to the RNA polymerase III promoter.

### Example 5

### Testing Plants Resistant to Geminivirus

The constructs of Examples 3 and 4 are transformed into a 4-6 week old N. benthamiana plant as described in Example 1. In particular, leaves of the N. benthamiana are infiltrated with Agrobacterium strain expressing each of the constructs. Plants are bred and those having constructs integrated into the genome are identified.

The identified individual plants are then challenged with geminivirus. Following infection, visual observation of geminivirus infection symptoms is undertaken, with scoring of leaf curling, chlorotic lesions, mosaic, malformation, size reduction and stunting. A scale of zero to four, with zero representing no observable symptoms and four representing severe symptoms, can be used.

After an individual plant is challenged, an uninfected plant is placed in proximity to the individual plant. The degree of geminivirus migration from the challenged plant to the uninfected plant can be ascertained by visual observation of geminivirus infection symptoms in the uninfected plant, as described above.

The claimed subject matter is not to be limited in scope by the specific embodiments described herein. Indeed, various modifications of the claimed subject matter in addition to those described herein will become apparent to those skilled in the art from the foregoing description. Such modifications are intended to fall within the scope of the appended claims.

All patents, applications, publications, test methods, literature, and other materials cited herein are hereby incorporated by reference in their entirety as if physically present in this specification.

1. A method for generating a plant cell having an increased resistance to a geminivirus infection, wherein the method comprises introducing into the genome of the plant cell:
(i) a first nucleic acid comprising a sequence encoding a Clustered Regularly Interspaced Short Palindromic Repeats (CRISPR)-associated site-specific nuclease protein, wherein said protein is capable of introducing a double stranded break in the genome of the geminivirus at a target site, and wherein the first nucleic acid further comprises a promoter directing expression of the protein in the plant cell, or the expression of the protein is directed by the promoter of (ii) or (iii),
(ii) a second nucleic acid comprising a sequence encoding one or more Clustered Regularly Interspaced Short Palindromic Repeats (CRISPR) RNA (crRNA), wherein each of said one or more crRNA comprises a sequence complementary to one or more target sequences within the geminivirus genome and wherein said second nucleic acid further comprises one or more promoters directing expression of said one or more crRNA in said plant cell, or the expression of the one or more crRNA is directed by the promoter of (i) or (iii) and
(iii) a third nucleic acid comprising a sequence encoding one or more trans-activating crRNA (tracrRNA), wherein said third nucleic acid further comprises one or more promoters directing expression of said one or more tracrRNA in said plant cell, or the expression of the one or more tracrRNA is directed by the promoter of (i) or (ii),
wherein said second and third nucleic acids can be optionally replaced by a single fourth nucleic acid encoding one or more single guide RNA (sgRNA), wherein each of said one or more sgRNA comprises a sequence complementary to one or more target sequences within the geminivirus genome and wherein said fourth nucleic acid further comprises one or more promoters directing expression of said one or more sgRNA in said plant cell, or the expression of the one or more sgRNA is directed by the promoter of (i),
wherein said first and/or second and/or third nucleic acid or said first and/or fourth nucleic acid further comprises a left homology arm sequence and/or a right homology arm sequence, each homology arm sequence comprising a nucleotide sequence from the geminivirus genome that is adjacent to the target site of the double stranded break introduced by the protein within a geminivirus target sequence, and wherein the left homology arm sequence and the right homology arm sequence are effective to introduce the nucleic acid sequences contained between them into the geminivirus genome at a geminivirus target sequence by homologous recombination, and
wherein said first, second and third nucleic acid or said first and fourth nucleic acid become stably integrated into the genome of the plant cell.

2. The method of claim 1, wherein the size of the nucleic acid sequence between the left homology arm sequence and the right homology arm sequence is such that upon homologous recombination into the geminivirus genome it prevents such genome from being packaged into viral particles and/or the geminivirus target sequence is a sequence of the geminivirus genome essential for the infection of a plant cell.

3. The method of claim 1, wherein the first and second nucleic acid, the first and third nucleic acid, the second and third nucleic acids, the first, second and third nucleic acid or the first and fourth nucleic acid are placed on a single nucleic acid molecule for introducing into the genome of the plant cell.

4. The method of claim 1, wherein upon exposure of the plant cell to a geminivirus,
(a) the site-specific nuclease protein mediates a double stranded break in the geminivirus genome at or near the geminivirus target site, and
(b) one or more of the first and/or second and/or third or one or more of the first and/or fourth nucleic acids are inserted into the geminivirus genome at the double stranded break by homologous recombination to form a modified geminivirus;
wherein the modified geminivirus is unable to undergo one or more of replication, packaging, transport from the plant cell, or infection of a plant cell.

5. The method of claim 1, wherein the left homology arm is at least 30 bp, optionally at least 400 bp, and the right homology arm is at least 30 bp, optionally at least 400 bp.

6. The method of claim 1, wherein the sequence contained within the target sequence is within a Rep protein ORF, a movement protein ORF, a coat protein protein ORF, a hairpin region that mediates replication origin, or a satellite DNA.

7. The method of claim 1, wherein the Clustered Regularly Interspaced Short Palindromic Repeats (CRISPR)-associated site-specific nuclease protein is a Cas protein, preferably Cas9 protein, or Cpf1 protein.

8. The method of claim 1, wherein one or more promoter is inducible in response to a geminivirus infection.

9. The method of claim 1, wherein the one or more crRNA or the one or more sgRNA comprises a sequence complementary to one or more target sequences within the genomes of more than one geminivirus species or more than one strain of the same geminivirus species.

10. The method of claim 1, wherein each homology arm sequence comprising a nucleotide sequence that is adjacent to the target site of the double stranded break introduced by the protein within target sequence, and wherein the left homology arm sequence and the right homology arm sequence are effective to introduce the nucleic acid sequences contained between them into the genomes of more than one geminivirus species or more than one strain of the same geminivirus species by homologous recombination.

11. The method of claim 10, wherein the generated plant cell having an increased resistance to infections of multiple geminivirus species or to infections of multiple strains of the same geminivirus species.

12. A method for generating a plant having an increased resistance to a geminivirus infection, wherein the method comprising
(I) generating a plant cell having an increased geminivirus resistance by a method of claim 1, and
(II) regenerating a plant from the plant cell of (I).

13. A plant cell generated by the method of claim 1.

14. A plant or plant part generated by the method of claim 12.

15. A vector comprising:
(i) a first nucleic acid comprising a sequence encoding a Clustered Regularly Interspaced Short Palindromic Repeats (CRISPR)-associated site-specific nuclease protein, wherein said protein is capable of introducing a double stranded break in the genome of a geminivirus at a target site, and wherein the first nucleic acid further comprises a promoter directing expression of the protein in a plant cell, or the expression of the protein is directed by the promoter of (ii) or (iii),
(ii) a second nucleic acid comprising a sequence encoding one or more Clustered Regularly Interspaced Short Palindromic Repeats (CRISPR) RNA (crRNA), wherein each of said one or more crRNA comprises a sequence complementary to one or more target sequences within the geminivirus genome and wherein said second nucleic acid further comprises one or more promoters directing expression of said one or more crRNA in a plant cell, or the expression of the one or more crRNA is directed by the promoter of (i) or (iii),
(iii) a third nucleic acid comprising a sequence encoding one or more trans-activating crRNA (tracrRNA), wherein said third nucleic acid further comprises one or more promoters directing expression of said one or more tracrRNA in a plant cell, or the expression of the one or more tracrRNA is directed by the promoter of (i) or (ii), and
(iv) a left homology arm sequence and/or a right homology arm sequence, each homology arm sequence comprising a nucleotide sequence from the geminivirus genome that is adjacent to the target site of the double stranded break introduced by the protein within a geminivirus target sequence, and wherein the left homology arm sequence and the right homology arm sequence are effective to introduce the nucleic acid sequences contained between them into a geminivirus genome at a geminivirus target sequence by homologous recombination.

16. The vector of claim 15, wherein the size of the nucleic acid sequence between the left homology arm sequence and the right homology arm sequence is such that upon homologous recombination into the geminivirus genome it prevents such genome from being packaged into viral particles and/or the geminivirus target sequence is a sequence of the geminivirus genome essential for the infection of a plant cell.

17. The vector of claim 15, wherein the left homology arm is at least 30 bp, optionally at least 400 bp, and the right homology arm is at least 30 bp, optionally at least 400 bp.

18. The vector of claim 15, wherein the sequence contained within the target sequence is within a Rep protein ORF, a movement protein ORF, a coat protein protein ORF, a hairpin region that mediates replication origin, or a satellite DNA.

19. The vector of claim 15, wherein the Clustered Regularly Interspaced Short Palindromic Repeats (CRISPR)-associated site-specific nuclease protein is a Cas protein, preferably Cas9 protein, or Cpf1 protein.

20. The vector of claim 15, wherein one or more of the promoters is inducible in response to a geminivirus infection.

21. The vector of claim 15, wherein the one or more crRNA comprises a sequence complementary to one or more target sequences within the genomes of more than one geminivirus species or more than one strain of the same geminivirus species.

22. The vector of claim 15, wherein each homology arm sequence comprising a nucleotide sequence that is adjacent to the target site of the double stranded break introduced by the protein within target sequence, and wherein the left homology arm sequence and the right homology arm sequence are effective to introduce the nucleic acid sequences contained between them into the genomes of more than one geminivirus species or more than one strain of the same geminivirus species by homologous recombination.

23. A vector comprising:
(i) a first nucleic acid comprising a sequence encoding a Clustered Regularly Interspaced Short Palindromic Repeats (CRISPR)-associated site-specific nuclease protein, wherein said protein is capable of introducing a double stranded break in the genome of a geminivirus at a target site, and wherein the first nucleic acid further comprises a promoter directing expression of the protein in a plant cell, or the expression of the protein is directed by the promoter of (ii),
(ii) a second nucleic acid encoding one or more single guide RNA (sgRNA), wherein each of said one or more sgRNA comprises a sequence complementary to one or more target sequences within the geminivirus genome and wherein said second nucleic acid further comprises one or more promoters directing expression of said one or more sgRNA in a plant cell, or the expression of the one or more sgRNA is directed by the promoter of (i), and
(iii) a left homology arm sequence and/or a right homology arm sequence, each homology arm sequence comprising a nucleotide sequence from the geminivirus genome that is adjacent to the target site of the double stranded break introduced by the protein within a geminivirus target sequence, and wherein the left homology arm sequence and the right homology arm sequence are effective to introduce the nucleic acid sequences contained between them into a geminivirus genome at a geminivirus target sequence by homologous recombination.

24. The vector of claim 23, wherein the size of the nucleic acid sequence between the left homology arm sequence and the right homology arm sequence is such that upon homologous recombination into the geminivirus genome it prevents such genome from being packaged into viral particles and/or the geminivirus target sequence is a sequence of the geminivirus genome essential for the infection of a plant cell.

25. The vector of claim 23, wherein the left homology arm is at least 30 bp, and the right homology arm is at least 30 bp.

26. The vector of claim 23, wherein the sequence contained within the target sequence is within a Rep protein ORF, a movement protein ORF, a coat protein protein ORF, a hairpin region that mediates replication origin, or a satellite DNA.

27. The vector of claim 23, wherein the Clustered Regularly Interspaced Short Palindromic Repeats (CRISPR)-associated site-specific nuclease protein is a Cas protein, preferably Cas9 protein, or Cpf1 protein.

28. The vector of claim 23, wherein one or more of the promoters is inducible in response to a geminivirus infection.

29. The vector of claim 23, wherein the one or more sgRNA comprises a sequence complementary to one or more target sequences within the genomes of more than one geminivirus species or more than one strain of the same geminivirus species.

30. The vector of claim 23, wherein each homology arm sequence comprising a nucleotide sequence that is adjacent to the target site of the double stranded break introduced by the protein within target sequence, and wherein the left homology arm sequence and the right homology arm sequence are effective to introduce the nucleic acid sequences contained between them into the genomes of more than one geminivirus species or more than one strain of the same geminivirus species by homologous recombination.

31. A plant, plant part, or plant cell that has increased resistance to geminivirus infection, comprising a vector of claim 15 or comprising stably integrated into the genome:
(i) a first nucleic acid comprising a sequence encoding a Clustered Regularly Interspaced Short Palindromic Repeats (CRISPR)-associated site-specific nuclease protein, wherein said protein is capable of introducing a double stranded break in the genome of the geminivirus at a target site, and wherein the first nucleic acid further comprises a promoter directing expression of the protein in at least one plant cell, or the expression of the protein is directed by the promoter of (ii) or (iii),
(ii) a second nucleic acid comprising a sequence encoding one or more Clustered Regularly Interspaced Short Palindromic Repeats (CRISPR) RNA (crRNA), wherein each of said one or more crRNA comprises a sequence complementary to one or more target sequences within the geminivirus genome and wherein said second nucleic acid further comprises one or more promoters directing expression of said one or more crRNA in at least one plant cell, or the expression of the one or more crRNA is directed by the promoter of (i) or (iii), and
(iii) a third nucleic acid comprising a sequence encoding one or more trans-activating crRNA (tracrRNA), wherein said third nucleic acid further comprises one or more promoters directing expression of said one or more tracrRNA in at least one plant cell, or the expression of the one or more tracrRNA is directed by the promoter of (i) or (ii),
wherein said second and third nucleic acids can be optionally replaced by a single fourth nucleic acid encoding one or more single guide RNA (sgRNA), wherein each of said one or more sgRNA comprises a sequence complementary to one or more target sequences within the geminivirus genome and wherein said fourth nucleic acid further comprises one or more promoters directing expression of said one or more sgRNA in at least one plant cell, or the expression of said one or more sgRNA is directed by the promoter of (i),
wherein said first and/or second and/or third nucleic acid or said first and/or fourth nucleic acid further comprises a left homology arm sequence and/or a right homology arm sequence, each homology arm sequence comprising a nucleotide sequence from the geminivirus genome that is adjacent to the target site of the double stranded break introduced by the protein within a geminivirus target sequence, and wherein the left homology arm sequence and the right homology arm sequence are effective to introduce the nucleic acid sequences contained between them into the geminivirus genome at a geminivirus target sequence by homologous recombination.

32. The plant, plant part, or plant cell of claim 31, wherein the size of the nucleic acid sequence between the left homology arm sequence and the right homology arm sequence is such that upon homologous recombination into the geminivirus genome it prevents such genome from being packaged into viral particles and/or the geminivirus target sequence is a sequence of the geminivirus genome essential for the infection of a plant cell.

33. The plant, plant part, or plant cell of claim 31, wherein upon exposure of the plant, plant part, or plant cell to a geminivirus,
(a) the site-specific nuclease protein mediates a double stranded break in the geminivirus genome at or near the geminivirus target site, and
(b) one or more of the first and/or second and/or third or one or more of the first and/or fourth nucleic acids are inserted into the geminivirus genome at the double stranded break by homologous recombination to form a modified geminivirus;
wherein the modified geminivirus is unable to undergo one or more of replication, packaging, transport from the plant cell, or infection of a plant cell.

34. The plant, plant part, or plant cell of claim 31, wherein the left homology arm is at least 30 bp, and the right homology arm is at least 30 bp.

35. The plant, plant part, or plant cell of claim 31, wherein the sequence contained within the target sequence is within a Rep protein ORF, a movement protein ORF, a coat protein protein ORF, a hairpin region that mediates replication origin, or a satellite DNA.

36. The plant, plant part, or plant cell of claim 31, wherein the Clustered Regularly Interspaced Short Palindromic Repeats (CRISPR)-associated site-specific nuclease protein is a Cas protein, preferably Cas9 protein, or Cpf1 protein.

37. The plant, plant part, or plant cell of claim 31, wherein one or more promoter is inducible in response to a geminivirus infection.

38. The plant, plant part, or plant cell of claim 31, wherein the one or more crRNA or the one or more sgRNA comprises a sequence complementary to one or more target sequences within the genomes of more than one geminivirus species or more than one strain of the same geminivirus species.

39. The plant, plant part, or plant cell of claim 31, wherein each homology arm sequence comprising a nucleotide sequence that is adjacent to the target site of the double stranded break introduced by the protein within target sequence, and wherein the left homology arm sequence and the right homology arm sequence are effective to introduce the nucleic acid sequences contained between them into the genomes of more than one geminivirus species or more than one strain of the same geminivirus species by homologous recombination.

40. The plant, plant part, or plant cell of claim 39, wherein the plant, plant part, or plant cell having an increased resistance to infections of multiple geminivirus species or to infections of multiple strains of the same geminivirus species.

41. A method for generating a plant cell having an increased resistance to a geminivirus infection, wherein the method comprises introducing into the genome of the plant cell:
(i) a first nucleic acid comprising a sequence encoding a Clustered Regularly Interspaced Short Palindromic Repeats-associated site specific nuclease protein, wherein said protein is capable of introducing a double stranded break in the genome of the geminivirus at a target site, and wherein said first nucleic acid sequence is operably linked to a promoter directing expression of the protein in said plant cell, or said first nucleic acid sequence is operably linked to the promoter of (ii) or (iii),
(ii) a second nucleic acid comprising a sequence encoding a Clustered Regularly Interspaced Short Palindromic Repeats (CRISPR) RNA (crRNA), wherein said crRNA comprises a sequence complementary to one or more target sequences within the geminivirus genome and wherein said second nucleic acid sequence is operably linked to a promoter directing expression of said crRNA in said plant cell, or said second nucleic acid sequence is operably linked to the promoter of (i) or (iii), and
(iii) a third nucleic acid encoding a trans-activating crRNA (tracrRNA), wherein said third nucleic acid sequence is operably linked to a promoter directing expression of said tracrRNA in said plant cell, or said third nucleic acid sequence is operably linked to the promoter of (i) or (ii),
wherein said second and third nucleic acids can be optionally replaced by a single fourth nucleic acid sequence encoding a hybrid crRNA tracrRNA (gRNA), wherein said gRNA comprises a sequence complementary to a target sequence within the geminivirus genome, and wherein said fourth nucleic acid sequence is operably linked to a promoter directing expression of said gRNA in said plant cell, or said fourth nucleic acid sequence is operably linked to the promoter of (i),
wherein said first and/or second and/or third nucleic acid or said first and/or fourth nucleic acid further comprises a left homology arm sequence and a right homology arm sequence, each homology arm sequence comprising a nucleotide sequence from the geminivirus genome that is adjacent to the target site of the double stranded break introduced by the protein within a geminivirus target sequence, and wherein the left homology arm sequence and the right homology arm sequence are effective to introduce the nucleic acid sequences contained between them into the geminivirus genome at a geminivirus target sequence by homologous recombination, and
wherein the said first, second and third nucleic acid or said first and fourth nucleic acid become stably integrated into the genome of the plant cell.

42. A method for generating a plant having an increased resistance to a geminivirus infection, wherein the method comprising
(I) generating a plant cell having an increased geminivirus resistance by a method of claim 41, and
(II) regenerating a plant from the plant cell of (I).

43. A plant cell generated by the method of claim 41.

44. A plant or plant part generated by the method of claim 42.

45. A vector comprising:
(i) a first nucleic acid comprising a sequence encoding a Clustered Regularly Interspaced Short Palindromic Repeats-associated site specific nuclease protein, wherein said protein is capable of introducing a double stranded break in the genome of the geminivirus at a target site, and wherein said first nucleic acid sequence is operably linked to a promoter directing expression of the protein in a plant cell, or said first nucleic acid sequence is operably linked to the promoter of (ii) or (iii),
(ii) a second nucleic acid comprising a sequence encoding a Clustered Regularly Interspaced Short Palindromic Repeats (CRISPR) R A (crRNA), wherein said crR A comprises a sequence complementary to one or more target sequences within the geminivirus genome and wherein said second nucleic acid sequence is operably linked to a promoter directing expression of said crRNA in a plant cell, or said second nucleic acid sequence is operably linked to the promoter of (i) or (iii),
(iii) a third nucleic acid encoding a trans-activating crRNA (tracrRNA), wherein said third nucleic acid sequence is operably linked to a promoter directing expression of said tracrRNA in a plant cell, or said second nucleic acid sequence is operably linked to the promoter of (i) or (ii), and
(iv) a left homology arm sequence and/or a right homology arm sequence, each homology arm sequence comprising a nucleotide sequence from the geminivirus genome that is adjacent to the target site of the double stranded break introduced by the protein within a geminivirus target sequence, and wherein the left homology arm sequence and the right homology arm sequence are effective to introduce the nucleic acid sequences contained between them into a geminivirus genome at a geminivirus target sequence by homologous recombination.

46. A vector comprising:
(i) a first nucleic acid comprising a sequence encoding a Clustered Regularly Interspaced Short Palindromic Repeats-associated site specific nuclease protein, wherein said protein is capable of introducing a double stranded break in the genome of the geminivirus at a target site, and wherein said first nucleic acid sequence is operably linked to a promoter directing expression of the protein in a plant cell, or said first nucleic acid sequence is operably linked to the promoter of (ii),
(ii) a second nucleic acid sequence encoding a hybrid crR A tracrR A (gRNA), wherein said gRNA comprises a sequence complementary to a target sequence within the geminivirus genome and wherein said second nucleic acid sequence is operably linked to a promoter directing expression of said gRNA in a plant cell, or said second nucleic acid sequence is operably linked to the promoter of (i), and
(iii) a left homology arm sequence and/or a right homology arm sequence, each homology arm sequence comprising a nucleotide sequence from the geminivirus genome that is adjacent to the target site of the double stranded break introduced by the protein within a geminivirus target sequence, and wherein the left homology arm sequence and the right homology arm sequence are effective to introduce the nucleic acid sequences contained between them into a geminivirus genome at a geminivirus target sequence by homologous recombination.

47. A plant, plant part, or plant cell that has increased resistance to geminivirus infection, comprising a vector of one of claim 45 or comprising stably integrated into the genome:
(i) a first nucleic acid comprising a sequence encoding a Clustered Regularly Interspaced Short Palindromic Repeats-associated site-specific nuclease protein, wherein said protein is capable of introducing a double stranded break in the genome of the geminivirus at a target site, and wherein said first nucleic acid sequence is operably linked to a promoter directing expression of the protein in at least one plant cell, or said first nucleic acid sequence is operably linked to the promoter of (ii) or (iii),
(ii) a second nucleic acid comprising a sequence encoding a Clustered Regularly Interspaced Short Palindromic Repeats (CRISPR) RNA (crRNA), wherein said crRNA comprises a sequence complementary to one or more target sequences within the geminivirus genome, and wherein said second nucleic acid sequence is operably linked to a promoter directing expression of said crRNA in at least one plant cell, or said second nucleic acid sequence is operably linked to the promoter of (i) or (iii), and
(iii) a third nucleic acid encoding a trans-activating crRNA (tracrRNA), wherein said third nucleic acid sequence is operably linked to a promoter directing expression of said tracrRNA in at least one plant cell, or said second nucleic acid sequence is operably linked to the promoter of (i) or (ii),
wherein said second and third nucleic acids can be optionally replaced by a single fourth nucleic acid sequence encoding a hybrid crRNA tracrRNA (gRNA), wherein said gRNA comprises a sequence complementary to a target sequence within the geminivirus genome and wherein said fourth nucleic acid sequence is operably linked to a promoter directing expression of said gRNA in at least one plant cell, or said fourth nucleic acid sequence is operably linked to the promoter of (i),
wherein said first and/or second and/or third nucleic acid or said first and/or fourth nucleic acid further comprises a left homology arm sequence and a right homology arm sequence, each homology arm sequence comprising a nucleotide sequence from the geminivirus genome that is adjacent to the target site of the double stranded break introduced by the protein within a geminivirus target sequence, and wherein the left homology arm sequence and the right homology arm sequence are effective to introduce the nucleic acid sequences contained between them into the geminivirus genome at a geminivirus target sequence by homologous recombination.

48. A method for generating a plant cell having an increased resistance to a geminivirus infection, wherein the method comprises introducing into the genome of the plant cell one nucleic acid molecule comprising:
(i) a first nucleic acid comprising a sequence encoding a Clustered Regularly Interspaced Short Palindromic Repeats-associated site specific nuclease protein, wherein said protein is capable of introducing a double stranded break in the genome of the geminivirus at a target site and wherein said first nucleic acid sequence is operably linked to a promoter directing expression of the protein in said plant cell, or said first nucleic acid sequence is operably linked to the promoter of (ii) or (iii),
(ii) a second nucleic acid comprising a sequence encoding a Clustered Regularly Interspaced Short Palindromic Repeats (CRISPR) RNA (crRNA), wherein said crRNA comprises a sequence complementary to one or more target sequences within the geminivirus genome and wherein said second nucleic acid sequence is operably linked to a promoter directing expression of said crRNA in said plant cell, or said second nucleic acid sequence is operably linked to the promoter of (i) or (iii), and
(iii) a third nucleic acid encoding a trans-activating crRNA (tracrRNA), wherein said third nucleic acid sequence is operably linked to a promoter directing expression of said tracrRNA in said plant cell, or said second nucleic acid sequence is operably linked to the promoter of (i) or (ii),
wherein said second and third nucleic acids can be optionally replaced by a single fourth nucleic acid sequence encoding a hybrid crRNA tracrRNA (gRNA), wherein said gRNA comprises a sequence complementary to a target sequence within the geminivirus genome and wherein said fourth nucleic acid sequence is operably linked to a promoter directing expression of said gRNA in said plant cell, or said fourth nucleic acid sequence is operably linked to the promoter of (i),
wherein said one nucleic acid molecule further comprises a left homology arm sequence and a right homology arm sequence, each homology arm sequence comprising a nucleotide sequence from the geminivirus genome that is adjacent to the target site of the double stranded break introduced by the protein within a geminivirus target sequence, and wherein the left homology arm sequence and the right homology arm sequence are effective to introduce the nucleic acid sequences contained between them into the geminivirus genome at a geminivirus target sequence by homologous recombination, and
wherein said one nucleic acid molecule becomes stably integrated into the genome of the plant cell.

49. A method for generating a plant having an increased resistance to a geminivirus infection, wherein the method comprising
(I) generating a plant cell having an increased geminivirus resistance by a method of claim 48, and
(II) regenerating a plant from the plant cell of (I).

50. A plant cell generated by the method of claim 48.

51. A plant or plant part generated by the method of claim 49.

52. A plant, plant part, or plant cell that has increased resistance to geminivirus infection, comprising stably integrated into the genome one nucleic acid molecule comprising:
(i) a first nucleic acid comprising a sequence encoding a Clustered Regularly Interspaced Short Palindromic Repeats-associated site specific nuclease protein, wherein said protein is capable of introducing a double stranded break in the genome of the geminivirus at a target site, and wherein said first nucleic acid sequence is operably linked to a promoter directing expression of the protein in at least one plant cell, or said first nucleic acid sequence is operably linked to the promoter of (ii) or (iii),
(ii) a second nucleic acid comprising a sequence encoding a Clustered Regularly Interspaced Short Palindromic Repeats (CRISPR) RNA (crRNA), wherein said crRNA comprises a sequence complementary to one or more target sequences within the geminivirus genome and wherein said second nucleic acid sequence is operably linked to a promoter directing expression of said crRNA in at least one plant cell, or said second nucleic acid sequence is operably linked to the promoter of (i) or (iii), and
(iii) a third nucleic acid encoding a trans-activating crRNA (tracrRNA), wherein said third nucleic acid sequence is operably linked to a promoter directing expression of said tracrRNA in at least one plant cell, or said second nucleic acid sequence is operably linked to the promoter of (i) or (ii),
wherein said second and third nucleic acids can be optionally replaced by a single fourth nucleic acid sequence encoding a hybrid crRNA/tracrRNA (gRNA), wherein said gRNA comprises a sequence complementary to a target sequence within the geminivirus genome and wherein said fourth nucleic acid sequence is operably linked to a promoter directing expression of said gRNA in said plant cell, or said fourth nucleic acid sequence is operably linked to the promoter of (i),
wherein said one nucleic acid molecule further comprises a left homology arm sequence and a right homology arm sequence, each homology arm sequence comprising a nucleotide sequence from the geminivirus genome that is adjacent to the target site of the double stranded break introduced by the protein within a geminivirus target sequence, and wherein the left homology arm sequence and the right homology arm sequence are effective to introduce the nucleic acid sequences contained between them into the geminivirus genome at a geminivirus target sequence by homologous recombination.
